(19) United States
(12) Patent Application Publication
(10) Pub. No.: US 2024/0266016 A1
(43) Pub. Date: Aug. 8, 2024

(54) AUTOMATED EXTRACTION AND UNIFICATION OF HEALTH RECORD RESOURCES THROUGH FAST HEALTHCARE INTEROPERABILITY RESOURCE CONVERSION

(71) Applicant: Nishigandha Mubin Mirkar, EDISON, NJ (US)

(72) Inventor: Nishigandha Mubin Mirkar, EDISON, NJ (US)

(21) Appl. No.: 18/411,112

(22) Filed: Jan. 12, 2024

Related U.S. Application Data
(60) Provisional application No. 63/482,978, filed on Feb. 2, 2023.

(51) Int. Cl. G16H 15/00; G16H 10/20; G16H 10/60

(52) U.S. Cl. CPC G16H 15/00; G16H 10/20; G16H 10/60

(57) ABSTRACT

A system automating extraction and unification of health data resources. Health data received from various sources is in the form of structured or unstructured health information. The system extracts health entities from unstructured data. Converts health resources in the various formats including FHIR-DSTU2, FHIR-STU3, Fhir-R4, C-CDA and Dicom image metadata to a common target format of FHIR R4. Highlight feature ‘Resource linking’ implements business intelligence through which it establishes connections amongst related health resources and generates unified health records. Allows integration and visualization of high quality Dicom images along with rest of the health data in one single platform. Supports global content search which allows user to enter any key word(s) and fetch all relevant health records spanning across various health data resources. The system also aims at maximum positive user experience through easy to use, intuitive, efficient and user centric User Interface design.

### CROSS REFERENCE TO RELATED APPLICATION(S)

The present application claims the priority of U.S. provisional patent application Ser. No. 63/482,978, filing date Feb. 2, 2023, inventor and applicant Nishigandha Mubin Mirkar.

### FIELD OF THE INVENTION

This invention relates to health data aggregation, analysis and presentation.

### BACKGROUND OF THE INVENTION

The majority of healthcare decisions are made based on health data. In today's world, this health data can originate from a variety of sources including EHR/EMR (wherein “EHR” stands for “electronic heath record”, and “EMR” stands for “electronic medical record”) systems, printed documents from health organizations, handwritten scripts, radiology CD/DVD (wherein “CD” stands for “compact disc” and “DVD” stands for “digital video disc”), wearable devices, registered IoMT (wherein “IoMT” stands for “internet of medical things) devices, laboratory tests, diagnostic procedures etc. All this data can be in either structured or unstructured format.

Structured health data may be in variety of formats including C-CDA (wherein “C-CDA” stands for “consolidated clinical document architecture”) and HL7 FHIR (wherein “HL7” is a registered trademark and “FHIR” stands for “fast healthcare interoperability resource”). FHIR is a standard for health care data exchange, published by HL7(registered trademark). FHIR(trademarked) supports RESTful webservices and open web technologies like XML and JSON (“JSON” stands for “JavaScript Object Notation”). REST stands for Representational state transfer. FHIR formats in the order of release date are DSTU2, STU3 and R4. One or more embodiments of the present invention is configured to be used to accommodate more advanced versions of FHIR as well.

As stated in U.S. Pat. No. 11,107,563, to Vogt et al., issued Aug. 31, 2021, which is incorporated herein by reference:

“FHIR is a standard for health care data exchange. The FHIR specification uses representational state transfer (REST) techniques to enable integration of a wide range of healthcare teams and organizations. FHIR is based on resources, each of which is defined by a uniform resource locator (URL), metadata, a set of defined data elements, and an extensibility framework.

Healthcare data is generated by doctors, hospitals, patients, and insurance companies, and is stored is [sic] an electronic health record (EMR). FHIR standardizes the format of the healthcare records and allows for data inter-change between the diverse entities that create, modify, and use health care data. The structure of FHIR structure definitions is stored in a FHIR structure definition, so FHIR is self-descriptive.” (Vogt et al., U.S. Pat. No. 11,107,563, col. 1, Ins. 19-34).

ONC's (“ONC” stands for “Office of the National Coordinator for Heath Information Technology”) Cures Act Final Rule supports seamless and secure access, exchange, and use of electronic health information. The ONC is an entity within the US Department of Health and Human Services.

ONC's Cures Act Final rule is designed to give patients and their healthcare providers secure access to health information. The ONC Cures Act Final Rule requires that certified health IT (information technology) developers update and provide their customers with FHIR-based application programming interfaces, also known as certified API (application programming interface) technology, by Dec. 31, 2022. In support of this requirement, many EHR/EMR provider systems have made the health data accessible over FHIR APIs. A majority of the EHR/EMR systems enabling data access via the APIs may provide data in either of the available FHIR formats: DSTU2, STU3 or R4.

Today a patient's health data is dispersed across many places like EHR/EMR systems, laboratory systems, paper copies in doctor's office, printed documents from health organizations, handwritten scripts, radiology reports, imaging disks (CD/DVD), wearable and IoMT devices etc. A patient cannot assume long term and desired access to data in EHR/EMR systems due to their respective terms and conditions. Few laboratory systems provide access to results via patient portal. Over the period of several years, a typical patient most likely would have visited multiple laboratory services across various geographical regions. Not all the laboratory data is accessible electronically or via EHR integrations. A portion of laboratory data may come from paper copies. There is a significant portion of care providers who still work without any EHR/EMR and prefer a traditional way of paper documents. In the United States, federal law requires care providers to maintain medical records for seven years from the date of service after which they may be deleted or made inaccessible.

Records obtained from a doctor's office, handwritten scripts, radiology reports, imaging CD/DVD, or printed documents from healthcare organizations all are in an unstructured format. Also, it is not easy to share radiology images with certain care providers. Unless the care provider has access to the PACs (“PAC” stands for “picture archiving and communication system”) imaging system of the radiology service provider, the only option patient has is to manually deliver the CD/DVD containing radiology images to the doctor's office. In addition to this it is important that a care provider's office should have a computing device equipped with a disk reader then only specific images can be viewed by the provider. Data generated by wearable devices and registered IoMT devices remain siloed. It can give valuable insights when connected with clinical health data.

While determining a treatment plan for a patient, all aspects of a patient's health history should be considered including current condition, progression, historical occurrences/treatments, risk factors, effectiveness etc. Instead of following a one solution fits all approach, an evidence based, and customized care plan should be developed.

A patient may receive treatments from different geographical locations at different times. Even across different countries in some cases. Individual specific unified health records accessible via portable device and owned by a patient himself/herself will be valuable in such scenarios. It is important to have a platform which will extract health information from various sources, convert them into a common format, link relevant pieces together and enable users to have access to unified health records specific to an individual.

This section is relating to health data in structured FHIR format. Data coming from different sources might contain information related to the same health data resources. For example, for health condition ‘Diabetes’, health records may exist in multiple EHR systems as a patient may have visited different care providers for the same condition. Also, some records might come from paper copy documents. Data ingested from all the sources needs to be processed accurately so that only one instance of ‘diabetes’ condition appears in the final unified health record of the patient and the rest of the details are accessible as linked records. Also, information for the same element may come from different health data resources. For example—information about medications received by a patient may come from resource MedicationOrder when fetched from EHR system exporting data in FHIR (trademarked) DSTU2 format or MedicationRequest resource when fetched from EHR system exporting data in FHIR (trademarked) STU3 or FHIR (trademarked) R4 format. Also, some data coming from R4 implementations may be in the form of FHIR resource ‘MedicationStatement’. Information from all these FHIR resources needs to be mapped accurately to a unified medication resource. There are several solutions out there providing aggregation of health data. In addition to the aggregation functionality, we need a system addressing the above-mentioned challenges.

In addition to the above we need a mechanism which will establish correct associations amongst FHIR resources of different types through resource linking. Even though HL7 (trademarked) FHIR (trademarked) definition of resources have a provision for adding references to other relevant FHIR resources, often, data received from EHR/EMR systems lacks these details. E.g., MedicationRequest received may not have reference to an encounter in which the medication was prescribed. Or the same MedicationRequest resource also may not have a reference to the exact reason/condition for which the medication was prescribed. Also, the same medication might have been prescribed as a part of two different encounters coming from two different EHR systems. All the relevant encounters/doctor's visits should be linked to the single unified medicine record along with necessary details like practitioners involved, conditions being treated, dosage, period etc. This will help generate a correct picture of exactly how many times specific medication has been taken by a patient, for what reasons it was prescribed, who prescribed it, dosage details, were there any allergic reactions and other relevant information.

Major EHR systems still do not have integration with PACS system where radiology service providers store DICOM (trademarked, wherein “DICOM” stands for “digital imaging and communications in medicine”, which is the international standard for medical images and related information) images. The only option patients currently have is to maintain multiple CD/DVD disks. It is important to have imaging data available in FHIR format along with the rest of the health data for a particular patient. Also, if DICOM images are available for viewing to the doctor at the time of treatment planning and diagnosis, it will help them make informed decisions. The system described in this disclosure enables users to view all medical, laboratory and imaging data through a single platform.

Over the period of a few years any individual's health data including EHR data, scanned documents and radiology images accumulates to a significantly large size. A system providing secure, compliant, and scalable storage mechanism for this health data accessible globally is much needed.

### SUMMARY OF THE INVENTION

One or more embodiments of the present invention provide a system, apparatus and/or method which allows users to share their health information with authorized family members, care providers, and researchers. Data available in the system can be shared with a researcher with a patient's consent. This can contribute to valuable health data pool and help in clinical studies.

One or more embodiments of the present invention are presented as a Software as a Service (SaaS) system which provides an automated mechanism allowing extraction, conversion and linking of FHIR resources resulting in unified health records.

This system is configured to receive health information from various sources in the form of structured or unstructured health data resources. The data sources can be EHR/EMR systems, paper copies of health records received from healthcare organizations, wearable/IoMT devices, old paper copies of prescriptions, handwritten doctor notes, lab reports, radiology exam reports and related DICOM(trademarked) images stored in CD/DVD disks. One or more embodiments of the present invention process unstructured health records received as text and DICOM (trademarked) images received as a part of radiology study through an extraction engine where the ingested data is converted into FHIR structured format. One or more embodiments of the present invention provide a system which also implements a feature “FHIR Converter Engine” allowing conversion of health data resources in the various formats including FHIR (trademarked) DSTU2, FHIR (trademarked) STU3, FHIR (trademarked) R4 and C-CDA (wherein “C-CDA” stands for “consolidated clinical document architecture) into a common target format of FHIR (trademarked) R4. Key feature of the system is ‘Resource linking’ implementing business intelligence through which it establishes connections amongst related FHIR resources. This helps achieve the goal of unified health records. One or more embodiments of the present invention provide a system that allows a user to bring in imaging data in the form of DICOM (trademarked) images, have it integrated into the unified health records and be able to visualize the high quality DICOM (trademarked) images along with rest of the health data in one single platform. Also, one or more embodiments of the present invention support global content search which allows a user to enter any key word(s) and fetch all relevant health records spanning across various health data resources in a single click. One or more embodiments of the present invention provide a system which is configured to be secure and compliant with necessary healthcare and data protection regulations including HIPPA (wherein “HIPPA” stands for “Health Insurance Portability and Accountability Act”) regulations.

One or more embodiments of the present invention provide a system that aims at maximum positive user experience through easy to use, intuitive, efficient and user centric user interface design.

### BRIEF DESCRIPTION OF THE DRAWINGS

FIG. 1 is a simplified diagram showing high level components and data flow in a health data extraction and unification method and system, in accordance with an embodiment of the present invention, wherein two types of data are processed structured and unstructured;

FIG. 2A is a simplified diagram showing a method for processing structured health data of the method and system shown in FIG. 1;

FIG. 2B is a simplified diagram showing a method for processing unstructured health data of the method and system shown in FIG. 1;

FIG. 3 is a simplified diagram showing an extraction engine used in unstructured data processing of the method and system shown in FIG. 1;

FIG. 4 is a simplified diagram showing the FHIR (trademarked) converter engine of the method and system shown in FIG. 1;

FIG. 5 is a simplified diagram showing the FHIR (trademarked) converter engine of FIG. 4 functionality through a generic example;

FIG. 6 is a simplified diagram showing a schema mapping function which is part of the FHIR (trademarked) converter engine of FIG. 4 using a real-world example;

FIG. 7 is a simplified diagram showing a resource linking feature of the FHIR (trademarked) converter engine of FIG. 4;

FIG. 8A is a simplified diagram showing health data in FHIR (trademarked) R4 format being submitted to resource linking component;

FIG. 8B is a simplified diagram showing one of the possible outcomes of the resource linking process initiated in FIG. 8A, wherein FIG. 8B version of the unified health record displays health data on the presentation layer when starting point is ‘Condition’ resource;

FIG. 8C is a simplified diagram showing one of the possible outcomes of the resource linking process initiated in FIG. 8A, wherein FIG. 8C version of the unified health record displays health data on the presentation layer when starting point is ‘MedicationRequest’ resource;

FIG. 8D is a simplified diagram showing one of the possible outcomes of the resource linking process initiated in FIG. 8A, wherein FIG. 8D version of the unified health record displays health data on the presentation layer when starting point is ‘Observation’ resource;

FIG. 9 is a simplified diagram showing FHIR (trademarked) converter engine functionality with example of condition resource;

FIG. 10 is a simplified diagram showing FHIR (trademarked) converter engine functionality with example of encounter resource;

FIG. 11 is a simplified diagram showing a process of DICOM (trademarked) image processing;

FIG. 12 shows a possible user interface design of ‘encounter’ i.e. healthcare visit resource;

FIG. 13 shows a possible user interface design of ‘condition’ resource;

FIG. 14 shows a possible user interface design of ‘Careplan’ resource;

FIG. 15A shows a possible user interface design of ‘Observation Type-Vital’ resource;

FIG. 15B shows another possible user interface design of “Observation Type-Vital” resource;

FIG. 16 shows a possible user interface design of ‘Document’ section;

FIG. 17 shows a possible user interface design of ‘imaging’ section with display of high-quality DICOM (trademarked) image along with metadata;

FIG. 18 shows a possible user interface design of ‘Medication’ and ‘immunization’ resources;

FIG. 19A shows a possible user interface design of ‘Global Search’ feature;

FIG. 19B shows another possible user interface design of ‘Global Search’ feature;

FIG. 20 shows a simplified diagram of system architecture of one or more embodiments of the present invention.

### DETAILED DESCRIPTION OF THE DRAWINGS

Various features of this invention are described in the sections below. Accompanying drawings will illustrate the various features of one or more systems, methods and/or apparatuses in accordance with one or more embodiments of the present invention. The description and drawings are illustrative of the invention and are not to be considered as limiting the invention. Specific details are described to provide a thorough understanding of various features of the present invention. However, in certain cases, well-known or conventional details are not described to provide a specific and concise discussion of the features of the current invention.

This disclosure is related to a software platform or system which automates extraction of health data and unification of health data resources through FHIR (trademarked) conversion. This system, in accordance with one or more embodiments of the present invention, can also be called “Health data extraction and unification system”. In the rest of this document, for the purpose of ease of reference and reading, the term “HealthRecord Application” will be used in place of “health data extraction and unification system”.

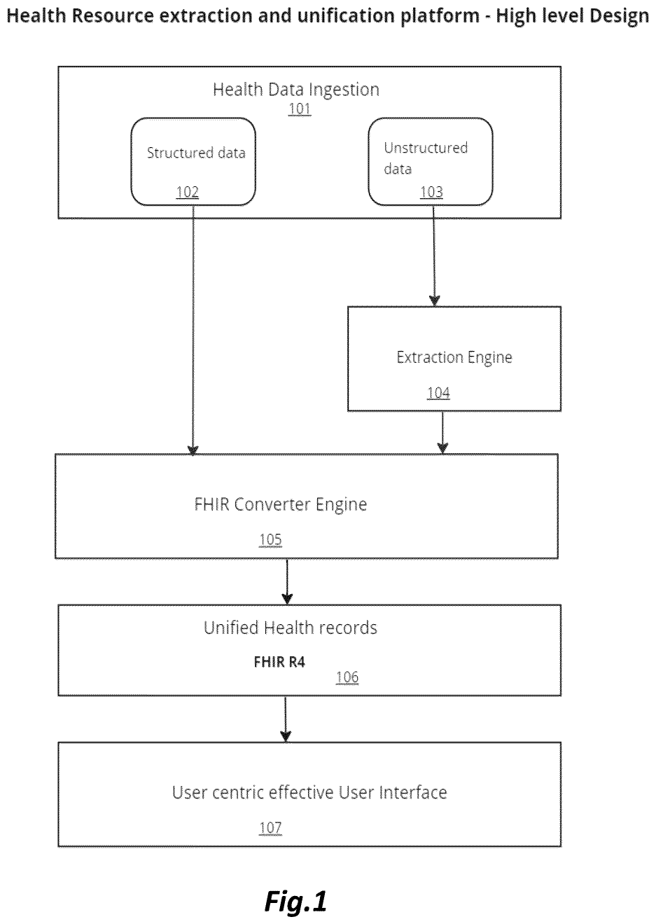

The “heath care data extraction and unification system” may include and/or may be implemented partially or completely by a one or more computers, having one or more computer memories, one or more computer processors, one or more computer input/output ports, one or more computer monitors, and one or more user computer interactive devices. The one or more computer memories may include computer software stored therein, which may be executed by the one or more computer processors to implement the system of one or more embodiments of the present invention. The user computer interactive devices may be any user computer interactive devices, such as touchscreen, computer mouse, and/or keyboard. The user centric effective user interface 107 in FIG. 1 may be implemented through a user interactive device and through computer software stored in the one or more computer memories, as executed by the one or more computer processors. The one or more computer input/output ports may be adapted to be connected for electronic communications to the internet. Any of the data or records referred to such as data 102, data 103, and unified health records 106, in FIG. 1 may be stored on one or more computer memories. Any of the engines referred to, such as engines 104 and 105, may be implemented by computer software stored in one or more computer memories and executed by one or more computer processors. Other abbreviations used in this disclosure are as per following.

EHR: Electronic Health records

EMR: Electronic Medical records

C-CDA: Consolidated clinical document architecture

FHIR: Fast Healthcare Interoperability Resources

OCR: Optical Character Recognition

API: Application programing interface

FIG. 1 shows a high-level configuration of health data extraction and unification system or “HealthRecord Application”. HealthRecord Application is configured to ingest data from a variety of sources. This ingested data could be in either structured or unstructured format. Structured data 102 is directly fed to FHIR (trademarked) converter engine 105. Structured data 102 may be mainly from but not limited to: (1) EHR/EMR systems through API integration; and (2) wearable devices and other IoMT devices generating health telemetry records. Since data received from these sources already has health entities identified, unlike unstructured data 103, structured data 102 does not go through extraction engine 104. Unstructured data 103 typically, this is data collected by a user. Unstructured data 103 could be received from variety of sources including but not limited to: (1) machine generated printed documents received from healthcare organization; (2) handwritten scripts, (3) laboratory reports, (4) radiology reports, (5) imaging CD/DVDs etc. Unstructured data 103 could have printed text, DICOM(trademarked) images or handwritten text. To get health entities from unstructured data 103, the unstructured data 103 is passed to the Extraction engine 104. Extraction engine 104 is where data passes through either of the two routes (1) OCR (“OCR” stands for “optical character recognition”) path or (2). DICOM (trademarked) path.

(1) OCR Path: Machine generated data received from healthcare organizations in the form of printed documents and handwritten scripts are scanned and uploaded into the HealthRecord Application. The scanned files go through OCR (Optical character recognition) engine where text is extracted. Further the extracted text passes through an analytics process enabled with artificial intelligence where health entities are detected. These health entities are further converted to structured FHIR (trademarked) format resulting into FHIR (trademarked) resources.

(2). DICOM (trademarked) Path: User can upload content of imaging CD/DVD to the system in the form of DICOM (trademarked) images. These images pass through extraction engine 104 where a key value pair data is extracted from DICOM (trademarked) images. This key value pair data is further converted to structured FHIR (trademarked) resources as ‘imagingStudy’. Also, the DICOM images are stored in long term, readily available and globally accessible storage locations, such as in one or more computer memories, and are configured to displayed as high-quality images through the presentation layer on one or more computer monitors.

FHIR (trademarked) converter engine 105, shown in FIG. 1, accepts structured health data which could be in any of the following formats: C-CDA, FHIR (trademarked) DSTU2, FHIR (trademarked) STU3 and FHIR (trademarked) R4. Also, this data could be sent as XML or JSON file. FHIR (trademarked) converter engine converts data from all these formats into a single standard format—FHIR (trademarked) R4 through schema mapping. After schema mapping, we get a set of independent and possibly disconnected FHIR (trademarked) R4 resources.

Currently there are several systems available out there which allow users to ingest and aggregate health data from different sources. These systems address only a portion of health data resources. Also, the data represented through these systems is often in the form of disconnected independent health information pieces. It is difficult to get a clear picture of the entire health of a patient. This implies that in addition to ingestion and aggregation of health data, we need a system which will correctly link all the resources together and display complete health information covering all spectrums of health.

In FHIR (trademarked) converter engine 105, through resource linking, health entities in FHIR (trademarked) R4 resource format are linked together which results in unified health records 106.

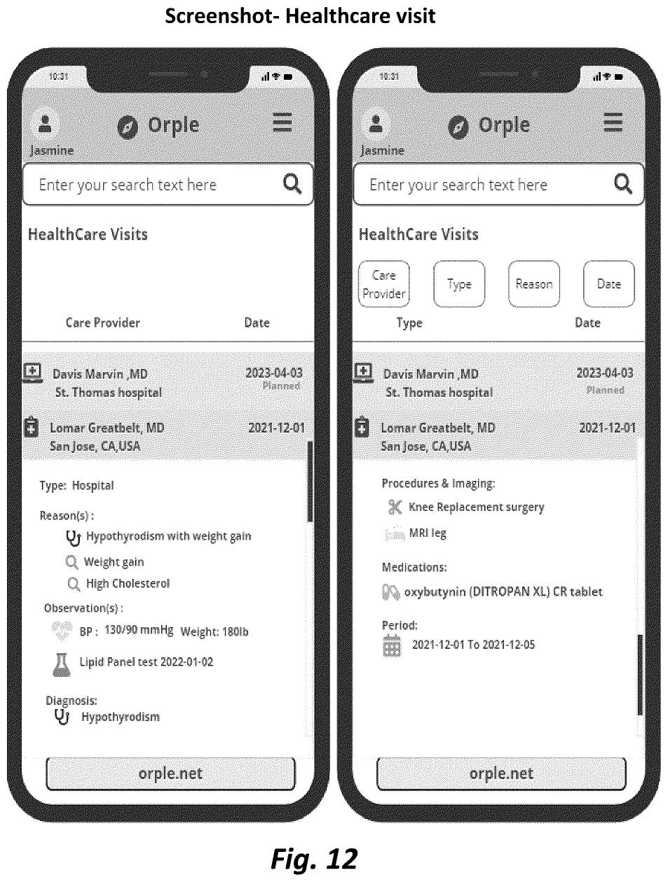

The structured health data resources in FHIR (trademarked) R4 format are available for export through API for external systems to consume. External research systems can consume these unified records and further enhance their study. Also, the system, method and apparatus of one or more embodiments of the present invention provides an enhanced user interface 107 which enables a user to view necessary health data in an intuitive and easy way. An example user Interface is shown in FIGS. 12-19.

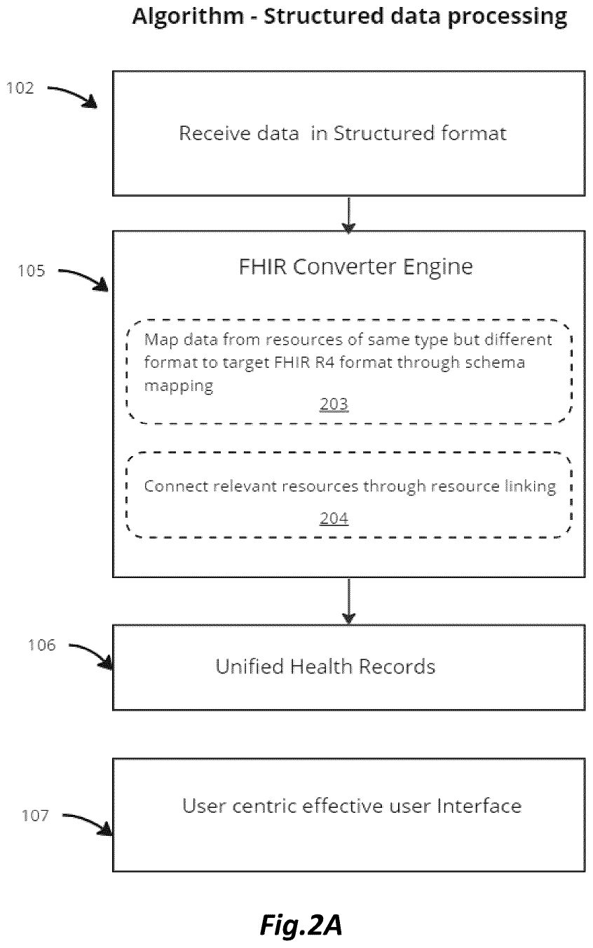

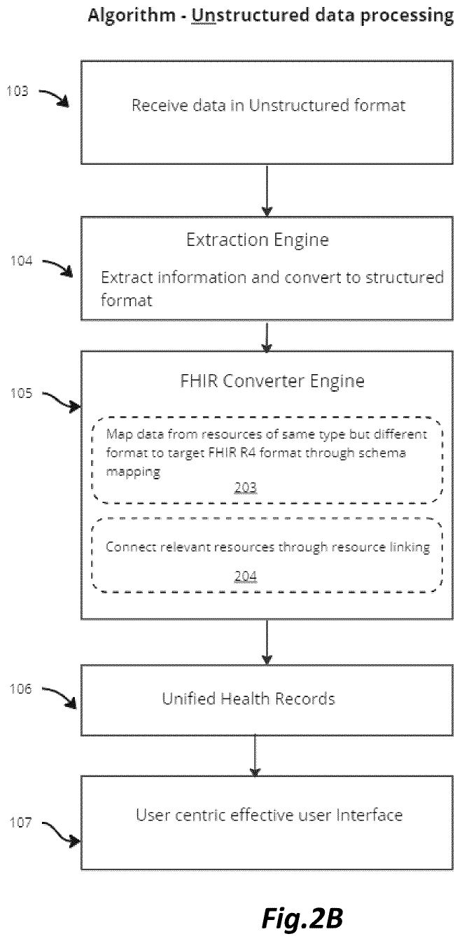

FIG. 2A shows a method for structured data processing. The HealthRecord Application mentioned in this disclosure can ingest structured health records from EHR/EMR system integration through APIs. In at least one embodiment, the structured health records, such as 102 are typically in xml or JSON files. Also, a system in accordance with one or more embodiments of the present invention is configured to ingest records from wearable devices and other IoMT devices through IoT connecter(s) The ingested data from all these sources can be, for example, in any of the following formats: C-CDA, DSTU (“DSTU” stands for “draft standard for trial use”) 2, STU (“STU” stands for “standard for trial use”) 3 and FHIR (trademarked) 4.

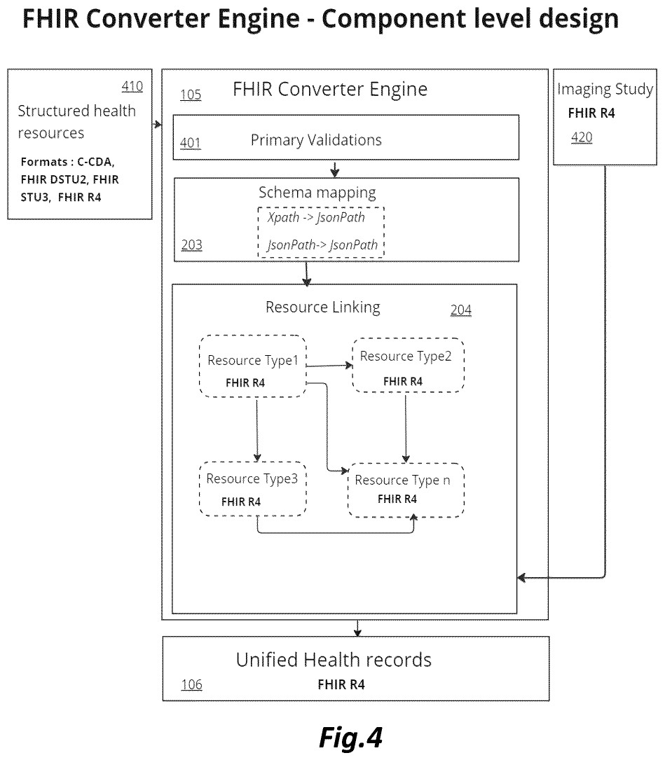

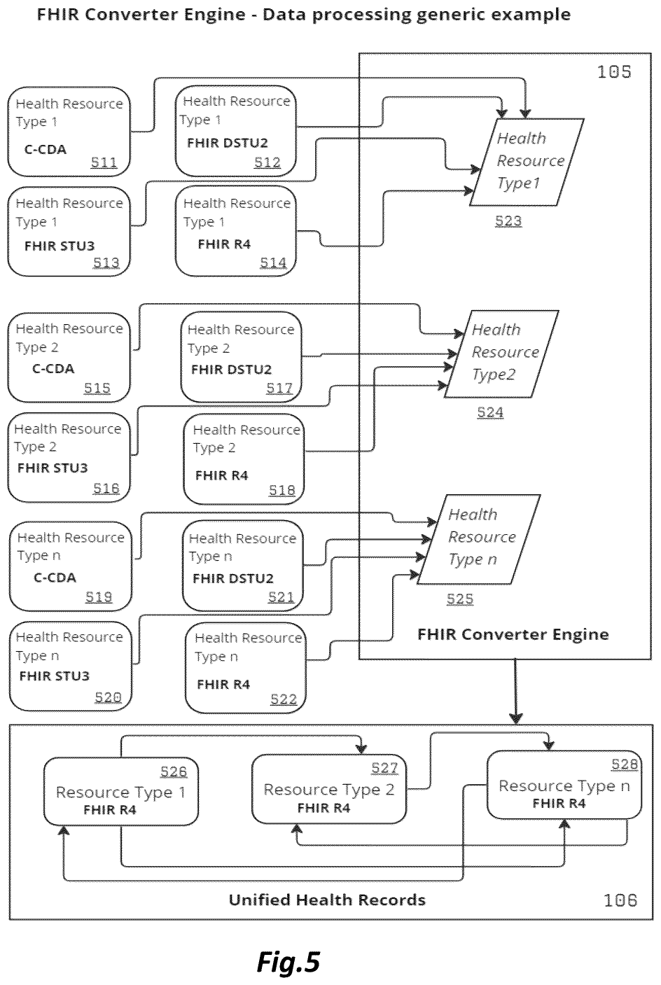

Most of this data received from EHR/EMR systems contains pieces of information which are missing references to other relevant health data resources. Also, data coming from multiple sources may contain duplicate data. All this information needs to be converted to a common format and linked together with accurate connections. For this purpose, the structured format data 102 received is sent to the FHIR (trademarked) converter engine 105 to achieve a unified health record. The FHIR (trademarked) converter engine 105 is explained in more detail in FIG. 4 and FIG. 5.

Inside the FHIR (trademarked) converter engine 105, health entities first pass through ‘schema mapping’ 203 shown in FIG. 2A. The schema mapping 203 maps data from entities of same type but different format to target FHIR (trademarked) R4 format. In this disclosure, health entities represented in structured format complying with either of the health data standards (C-CDA, DSTU2, STU3 or R4) are referred to as resources. In the rest of this document, we will be using the term ‘resources’ to represent health entities in structured format. Further, component 204 connects relevant resources in FHIR (trademarked) R4 format through resource linking. The outcome of this process is a unified health record 106 which can be consumed by a presentation layer and displayed in a user centric effective user interface 107, such as including on one or more computer monitors.

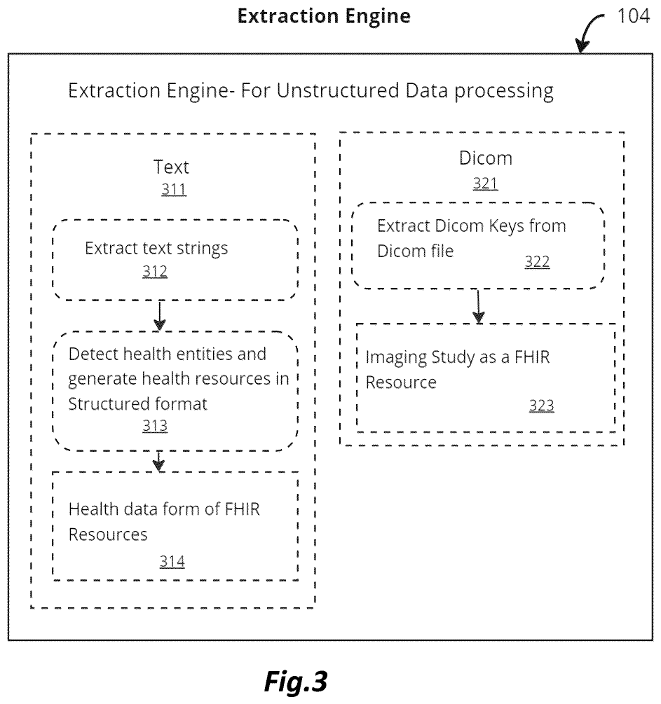

FIG. 2B shows a method for unstructured data processing. HealthRecord Application is configured to receive unstructured data 103 submitted by user. This data 103 can be in the form of but not limited to printed papers received from healthcare organizations, handwritten scripts, laboratory reports, imaging reports or DICOM (trademarked) images in CD/DVD received from radiology service provider. First, health data are extracted from all this information at step 104 as part of health data extraction engine. Operations of health extraction engine are explained in detail in FIG. 3. Extracted information from component 104 is submitted to FHIR (trademarked) converter engine 105 where data is passed through schema mapping module 203 and resource linking module 204. FHIR (trademarked) converter engine 105 is explained in more detail. In FIG. 4 and FIG. 5. The output of the FHIR (trademarked) converter engine 105 is unified health records 106. These records are configured to be processed by a presentation layer and displayed in user centric effective user interface 107.

FIG. 3 represents extraction engine 104 used during unstructured data processing.

A user can submit the data from paper copies in the form of scanned files. File formats for scanned files can be JPEG/JPG, PNG, BMP, TIFF, PDF, Word (DOCX), Excel (XLS), PowerPoint (PPT), and HTML etc. Additional formats can also be supported. These scanned files are submitted to cognitive services where through artificial intelligence methods, text, images, and tables are extracted from received data at step or component 312 shown in FIG. 3. The extracted information is further processed through health analytics—artificial intelligence methods which identify health data related pieces of information and convert them into structured health entities 313. The extracted health entities 313 are further converted to structured FHIR (trademarked) R4 resources 314.

The HealthRecord Application described in this disclosure accepts the DICOM (trademarked) images submitted by a user from the presentation layer. These images are large. Submitted images are stored in secure, long-term storage, such as long-term computer memory storage The system mentioned in one or more embodiments of the present invention enables a user to access these DICOM (trademarked) images in the form of high-quality images along with their meta data through its presentation layer. This eliminates the need for a patient/user having to carry and maintain several CD/DVD disks and relying on the care provider's ability to be able to review them based on the type of computer hardware available in the doctor's office. This will be a huge help for patients seeking treatment for health problems where diagnosis and treatment can be improved through analysis of imaging exams done in the past. A patient would not have to carry heavy binders of paper copies and CD/DVDs during their visits to the care provider's office. Also, this will reduce the efforts and stress for patient and their families specially when patient is suffering from a serious health condition.

DICOM(trademarked) images are sent to custom developed DICOM (trademarked) analysis service component 322. As part of component 322, all the key information and metadata is extracted from the DICOM (trademarked) images. Further this metadata is converted into structured FHIR (trademarked) R4 resource ‘imaging study’ 323.

FIG. 4 shows a component level design of the FHIR (trademarked) converter engine 105. FHIR (trademarked) converter engine 105 provides functionality to generate unified health records from the data received.

In FHIR (trademarked) converter engine 105, the first component 401 is the primary validation unit. To enhance the accuracy of the FHIR (trademarked) converter engine 105, various validations are performed. Such validations are customized per resource type and data structure type. E.g., matching record check to avoid duplication or verify if the practitioner mentioned in the record is already present in the system.

Apart from primary validation unit 401, FHIR (trademarked) converter engine 105 includes two further components (1) Schema mapping 203 and (2) resource linking204.

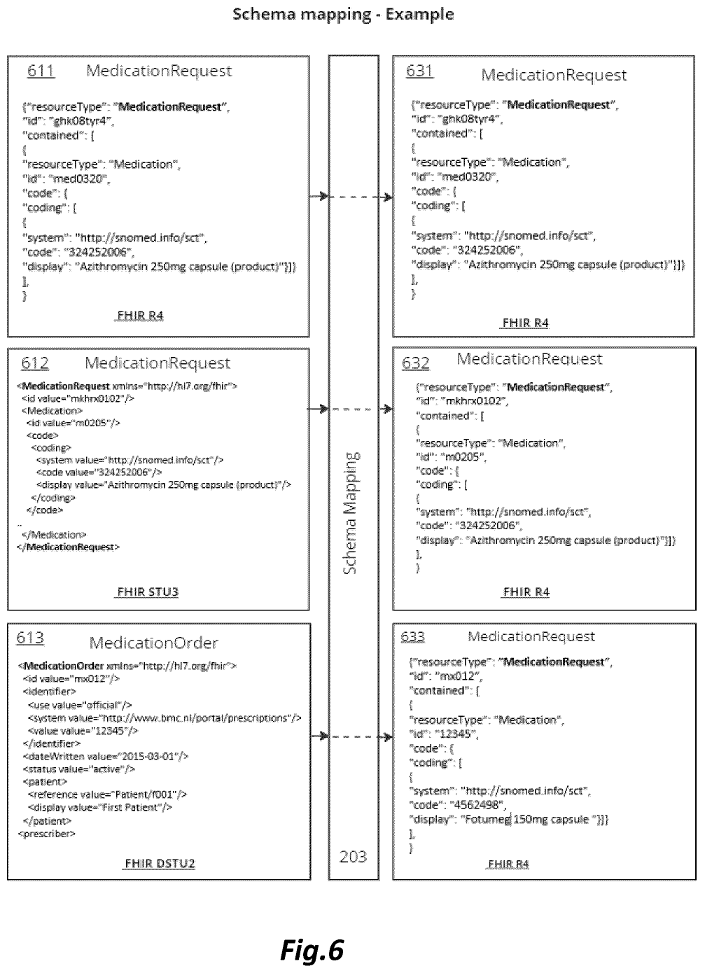

Schema mapping 203 is explained with an example in FIG. 6. In schema mapping module, data from various sources is received by FHIR (trademarked) converter engine 105 in the following formats: C-CDA, DSTU2, STU3, R4. In schema mapping module 203, records for a specific resource received in various formats are converted into a single target format—FHIR (trademarked) R4.

In an example scenario, four records for a condition resource might be submitted to schema mapping unit 203. Two records in xml files and two records in JSON files. The goal is to get all records in FHIR (trademarked) R4 JSON format so that they will be used in resource linking module 204.

To achieve the desired result, ingested data is processed by utilizing schema mapping rules in component 203 pre-defined in computer memory for each resource type. Using this schema mapping, the FHIR (trademarked) converter engine 105 decides where to get data from for each element in the target FHIR (trademarked) R4 data structure. In the context of above example, if FHIR (trademarked) converter engine 105 is processing incoming xml in FHIR (trademarked) DSTU2 format for condition resource, then to get the value for date when condition was recorded, it uses following mapping:

| | | | Target | | |
|---|---|---|---|---|---|
| Resource | Field | SourceType | Type | SourcePath | TargetPath |
| Condition | recordedDate | DSTU2 | R4 | Resource/ | Resource/ |
| | | | | dateRecorded | recordedDate |

Whereas, when processing health condition record extracted from C-CDA xml document, recordedDate can be read from following path inside problemSectionNode—[entry/act/effectiveTime/low]. The same corresponds to following schema mapping:

| | | Source | Target | | |
|---|---|---|---|---|---|
| Resource | Field | Type | Type | SourcePath | TargetPath |
| Condition | recordedDate | C-CDA | R4 | entry/act/ | Resource/ |
| | | | | effectiveTime/low | recordedDate |

In similar way, for processing STU3 and R4 condition resources, following schema mapping can be used:

| Resource | Field | SourceType | TargetType | SourcePath | TargetPath |
|---|---|---|---|---|---|
| Condition | Recorded | STU3 | R4 | Resource/ | Resource/ |
| | Date | | | assertedDate | recordedDate |
| Condition | Recorded | R4 | R4 | Resource/ | Resource/ |
| | Date | | | recordedDate | recordedDate |

After schema mapping 203, generated FHIR (trademarked) R4 resources are sent to Resource Linking Unit 204.

Data arrives at the FHIR converter engine 105 in the form of several disconnected pieces of health information. To make this data useful, all these pieces need to be connected and represented as unified health records. In an example scenario, a patient suffering from diabetes for more than ten years might have health information stored in several places. Few healthcondition resource records might exist in EHR/EMR system of recent care providers whereas some may come from handwritten assessment notes made by a doctor twelve years ago. Similarly, a portion of medication records may come from EHR/EMR and some from printed prescriptions. Allergic reaction to a medication given five years ago may come from ER visit hospital notes uploaded as printed/scanned document. Imaging data for the scanning done during the hospital visit might exist on a CD/DVD. Some recent laboratory test reports may be available through EHR/EMR and some as printed documents. Whereas most recent vitals related to pulse and blood sugar may be received from wearable devices. Alarming changes in vitals monitored through devices might have led to a change in care plan and medications during recent doctor's visit.

All the above-mentioned health data even though available in structured format will not be very useful unless correctly connected to each other. To get a clear idea about progression of a health condition, treatments given so far and determining next steps, it is important to connect the conditions with related healthcare visits and other relevant resources. Correct correlation must be established amongst resources. For example, Healthcare visit must be correctly linked to reason for the visit, observations made, vitals, allergic reactions, Careplan, medications, imaging etc. Additionally, medication resources should be connected to reason, condition, practitioner, encounter, and other relevant resources. Such a process when applied across all resource types, will result into unified health record, to be stored in one or more computer memories. In above mentioned example, unified health record would exactly tell you how many and which doctors the patient has been treated by for the given condition of diabetes. What all medications were given, with the aging body and disease progression, were there any allergic reactions to the treatment? What tests were performed to confirm the diagnosis? Can accurate laboratory reports and radiology images be shown to the doctor to help them understand the situation? Can the doctor see the trend in vitals—BP (blood pressure) and sugar level and validate the recently changed treatment?

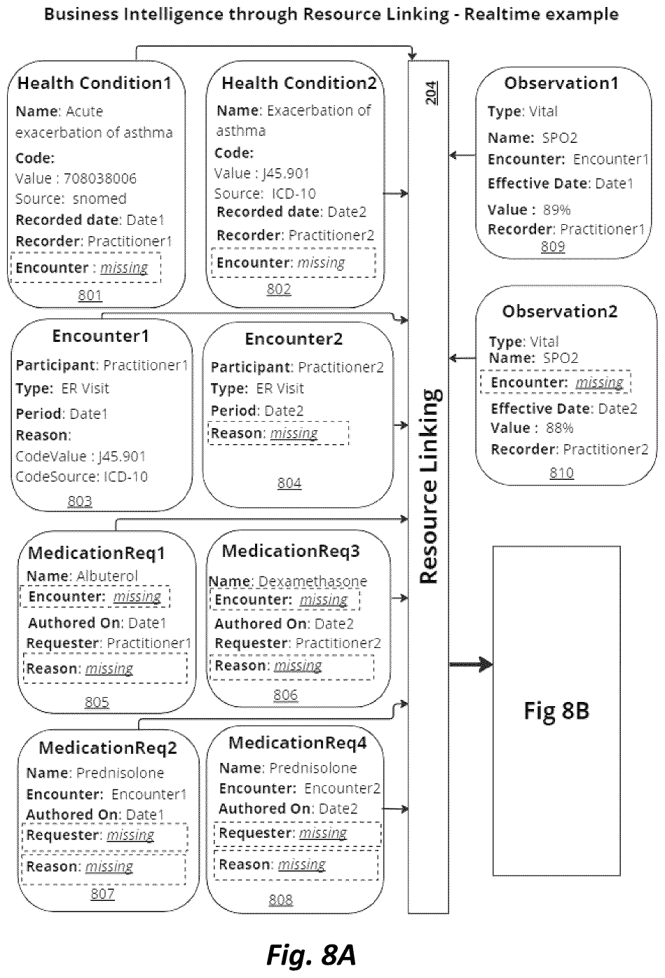

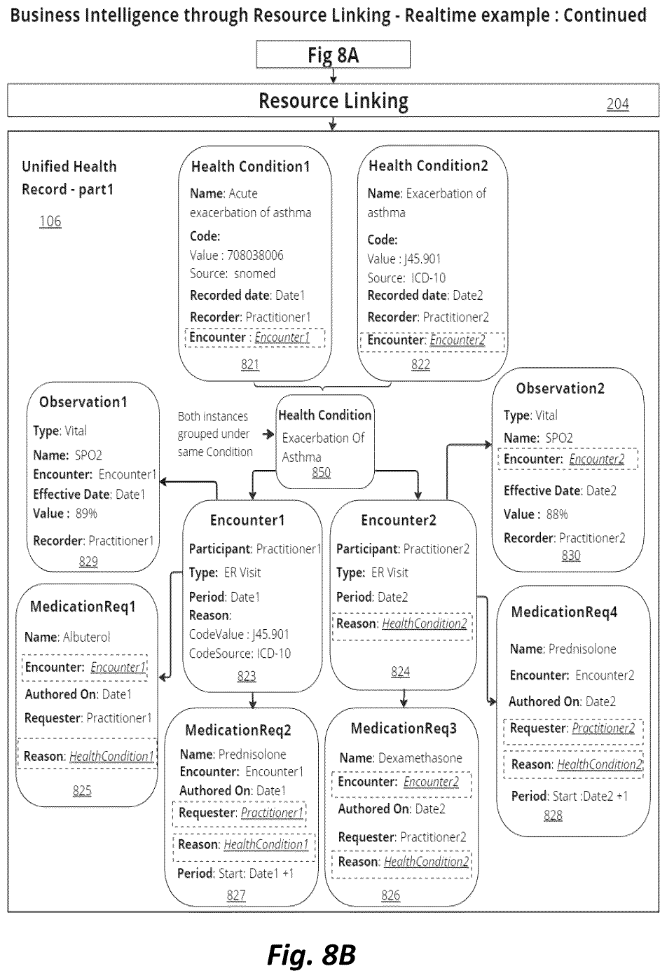

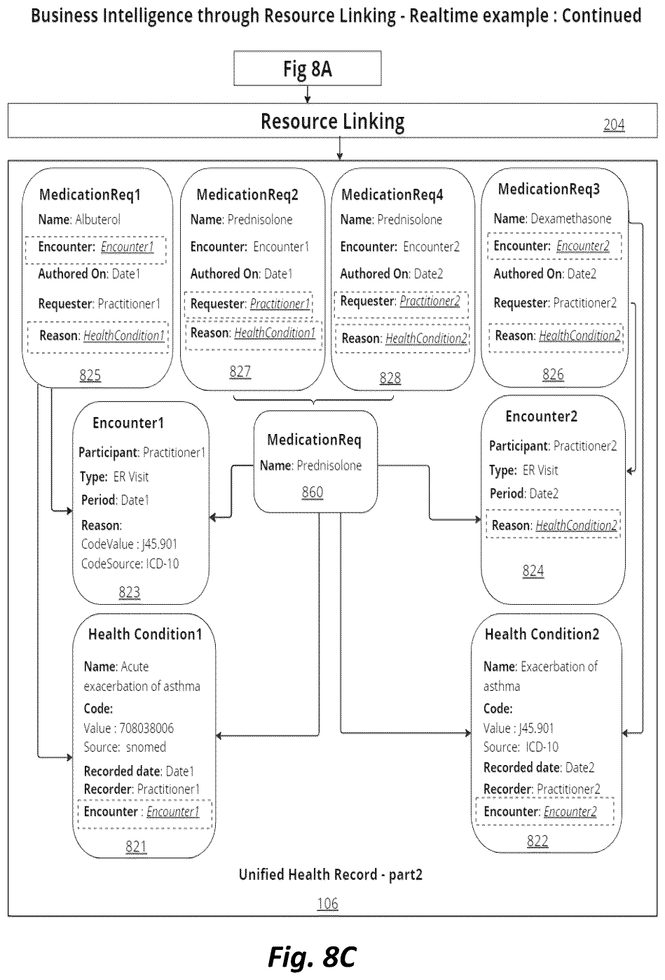

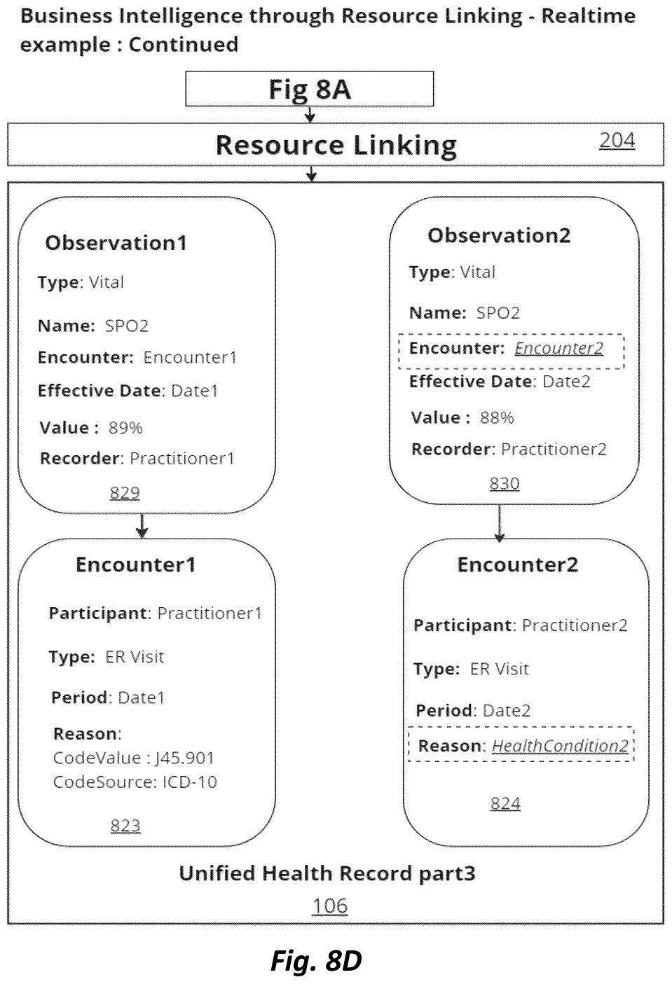

‘Resource linking’—204 feature, shown in FIG. 2B, is designed to address these challenges. It is described in more detail in FIGS. 8, 8A-D. In FIG. 4, component 204 identifies the Resource linking feature. The resource linking feature 204 causes, resources of different types to be linked together. This establishes meaningful relationships among various health data resources.

FIG. 5 shows the operation of FHIR (trademarked) converter engine 105 through an example. Components 511-522 represent data submitted to the FHIR (trademarked) converter engine 105 as structured health entities in formats—C-CDA, FHIR (trademarked) DSTU2, FHIR (trademarked) STU3 and FHIR (trademarked) R4 resources. This data is specific to certain health data resource types e.g., health condition, encounter, observation, medication etc. FHIR (trademarked) converter engine 105 processes the received data, converts all records into standard known FHIR (trademarked) R4 format. Components 511-514 represent health data resources for ResourceType1 each of different format (C-CDA, DSTU2, STU3 or R4) all of which are converted to equivalent FHIR R4 resources 523. Similarly, components 515-518 represent health data resources for ResoureType2 each of different format (C-CDA, DSTU2, STU3 or R4) and they are converted to equivalent FHIR (trademarked) R4 resources 524.

Further the FHIR (trademarked) converter engine 105 connects resources of various types together. The outcome of this process is Unified Health Records represented by component 106, which may be stored in one or more computer memories.

FIG. 6 describes schema mapping with an example. Three resources are submitted to the schema mapping unit 203—MedicationOrder in DSTU2 format (component 613), Medication Request in STU3 format (component 612) and MedicationRequest in R4 format (component 611). With the newer version STU3 of FHIR (trademarked), the MedicationOrder resource was replaced by MedicationRequest resource. The schema mapping unit 203 is programmed by computer software stored in computer memory and implemented by one or more computer processors to convert all these received resources into equivalent FHIR (trademarked) resources as MedicationRequest in FHIR (trademarked) R4 format represented by components 631, 632 and 633.

The resource linking unit 204 is a main feature of one or more embodiments of the present invention. FIGS. 8A-D show the operation of resource linking 204 with a generic example. As explained above, data received by FHIR (trademarked) converter engine 105 comes in the form of several disconnected pieces of health information. Even when received in common FHIR (trademarked) R4 format, often the data received by resource linking unit 204 lacks important references to other relevant resources. Plus, information received from other sources like devices or scanned files results in a siloed pool of health data resources. All these pieces need to be accurately connected to be able to derive value from the information. Just having aggregated condition resources or medication resources is not going to be enough for care providers to make informed and evidence-based decisions. The resource linking 204 unit feature plays a critical role in establishing these connections.

Resource linking 204 provides business intelligence through a custom developed rule engine covering a wide spectrum of HL7 FHIR (trademarked) resources. HL7 FHIR (trademarked) has defined standards for FHIR (trademarked) resources to represent different pieces of health information. Condition resource represents health problems, concerns. Encounter resource represents health care visits of multiple types—hospitalization, ER visit, in-office visit, laboratory visits, imaging exam, surgery, procedure etc. Observation resource represents observations of types—vital, lab, social history, or examination. Likewise, there are other resources to address each piece in the healthcare process. As per HL7 FHIR (trademarked) standard, each resource has unique structure and constraints. Resource linking 204 provides intelligence, through computer software programming stored in computer memory and implemented by a computer processor, which performs extensive analysis through its rule engine. The resource linking unit ensures that each FHIR (trademarked) resource is correctly connected to the other relevant resources in compliance with HL7 FHIR (trademarked) R4 definitions. The intelligence, as programmed by computer software, provided by this feature enables the system to establish relationships amongst resources of different types and different origins. It also provides the ability to track original data and changes for audit and verification purposes.

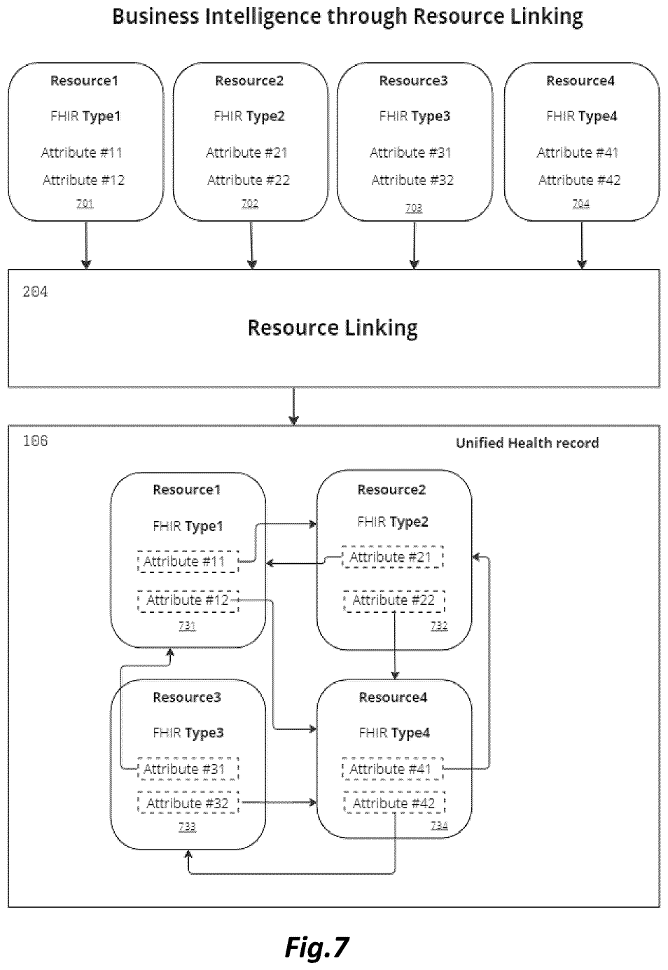

FIG. 7 shows that four different resources of distinct types are submitted to resource linking component 204. Each of these resources have sample attributes. These attributes could be a property representing unique information about the resource or reference to another resource. For e.g., a condition resource could have an attribute ‘name’ which would represent name of the health condition and it also could have another attribute ‘encounter’ which refers to doctor's office visit during which the condition was assessed, confirmed, or denied. Similarly, MedicationRequest resource could have an attribute for medication code which uniquely identifies the medication in reference to the pre-established medical information sources like SNOMED-CT (wherein “SNOMED-CT” stands for “systemized nomenclature of medicine—clinical terms”, which are standardized international, multilingual core collection of clinical healthcare terminology that can be used in electronic health care records) or ICD (wherein “ICD” means “international classification of diseases”) and another attribute which identifies reason for the medication. Resource linking feature 204 establishes connections between these attributes such as medication resource's reason attribute is connected to a condition resource and condition resource's encounter attribute is connected to an encounter resource. The outcome of this process is Unified health record 106. The unified health record 106 shows that attributes of different resources are connected to each other. This will enable a user to fetch relevant records while accessing data from the presentation layer.

Today, health data received from multiple sources like EHR/EMR, wearable/IoMT devices, scanned copies, lab work and radiology images etc. poses various challenges. A few of them are listed as below:

(1) Missing data for key references: Even though HL7 FHIR (trademarked) definitions of resources have provision to list referenced resources, often data received from EHR/EMR systems lacks these references. For example, condition resource has provision to declare related encounter and care providers; encounter resource has provision to declare participating practitioners and reason for the encounter; MedicationRequest resource has provision to declare reason and encounter during which medication was prescribed. Still, the data received has these important references missing from the FHIR (trademarked) resources. This reduces the usefulness of the information received through such FHIR (trademarked) resources.

(2) Variable coding: exact same piece of information related to certain FHIR (trademarked) resources received from different sources have different codes specified on them. E.g., Condition resource for “Diabetes Mellitus” may have a code specified as 73211009 source—SNOWMED whereas the same condition mentioned on medication record as a reason might have code specified as 45636-8 Source Loinc. This makes record correlation challenging.

(3) Inconsistent naming: Like previous item, the exact same piece of information related to certain FHIR (trademarked) resources received from different sources may have different names specified on them. E.g., Condition resource for ‘Diabetes Meillitus’ may have name specified Diabetes Mellitus whereas same condition mentioned on encounter record as a reason might have name specified ‘Diabetes Mellitus (DM Disorder)’. This creates inconsistencies in the information stored in FHIR (trademarked) resources.

(4) Missing data entry on records having same field: Due to the nature of HL7 FHIR (trademarked) resource definition, sometimes a piece of information can be entered in multiple places. However, due to insufficient training or any other reason, the person entering information may enter the data only in one place. This leads to incomplete records. For example, ‘note’ attribute can be added on both encounter and condition resource. If during data entry, only encounter's note was updated and not condition's, it would result into an incomplete condition record. If a condition was assessed as a part of an encounter and an important diagnosis was noted only in encounter's notes, it would not give important information stored in the note to the person reviewing data generated through condition record.

(5) Partially available health records: HL7 FHIR (trademarked) standards are evolving. In some situations, current definitions of FHIR (trademarked) resources do not provide required provision to enter necessary information for a specific resource. Example—encounter for a specific health condition has no way to directly specify imaging or laboratory exams done. A medication request resource has no way to specify if any allergic reaction has occurred.

All above challenges and more are addressed through resource linking unit 204. In here, FHIR (trademarked) resources are updated in one or more computer memories with necessary references to related resources, correct connections are established between FHIR (trademarked) resources so that critical piece of information can be accessed through referenced resources and other additional information can be made accessible through single unified record.

FIGS. 8A-D explain how records are processed through the resource linking unit—204 with a real-world example. The example shows only a portion of a patient's health records. The patient is a pediatric patient and has health condition ‘asthma’ and has had multiple admissions to emergency room due to asthma flareup also known as acute exacerbation of asthma.

FIG. 8A shows health data resources being submitted to the resource linking unit 204.

Health data resources being submitted to resource linking include the following types of records, shown in FIG. 8A:

Component 801: A condition record Health condition1 with missing reference to encounter resource.

Component 803: A visit/encounter record Encounter1 related to the Health Condition 1 with condition code having different value and source different than the actual condition record's code source.

Component 802: A condition record Health Condition2 with missing reference to encounter resource.

Component 804: A visit/encounter record Encounter2 related to the Health Condition2 with missing reference to the actual condition resource.

Component 805: MedicationRequest record MedicationReq1 related to Encouter1 with missing reference to encounter and condition (reason) resources.

Component 807: MedicationRequest record MedicationReq2 related to Encouter1 with missing reference to practitioner and condition resources.

Component 806: MedicationRequest record MedicationReq3 related to Encouter2 with missing reference to encounter and condition resources.

Component 808: MedicationRequest record MedicationReq4 related to Encouter2 with missing reference to practitioner and condition resources.

Component 809: Observation1 record of type Vital. Related to Encounter1 803.

Component 810: Observation2 record of type Vital. Related to Encounter2 804 with missing link to encounter resource.

Inside Resource Linking unit 204, the data undergoes deep analysis. Necessary updates are made to each resource record through the rule engine defining processing logic per resource Type. Outcome of the process is shown in FIG. 8B.

As part of the resource linking process, below are the changes made to each resource. Referring to FIG. 8B:

Component 821 shows that Health Condition1 is updated in one or more computer memories with a reference to Encounter1 resource.

Component 822 shows that Health Condition2 is updated in one or more computer memories with a reference to Encounter2 resource.

Component 824 shows that Encounter2 is updated in one or more computer memories with a reference to Health Condition2 822 resource.

Component 825 shows that MedicationReq1 is updated in one or more computer memories with a reference to Health Condition1 821 and Encounter1 823 resources.

Component 827 shows that MedicationReq2 is updated in one or more computer memories with a reference to Practitioner1 and HealthCondition1 821 resources.

Component 826 shows that MedicationReq3 is updated in one or more computer memories with a reference to Encounter2 and HealthCondition2 822 resources.

Component 828 shows that MedicationReq4 is updated in one or more computer memories with a reference to Practitioner2 and HealthCondition2 822 resources.

Component 830 shows that Observation2 is updated in one or more computer memories with a reference to Encounter2 824 resource.

These updates result into unified health records 106, which are stored in one or more computer memories These health records can be viewed in different ways through presentation layer (computer software application interface) on one or more computer monitors. FIGS. 8B-D show different ways health information can be viewed from the same record set.

FIG. 8B shows ‘Condition View’ i.e., the way in which information can be displayed, when user navigates to condition record in presentation layer, shown on a computer monitor.

Resource Linking unit 204 determines that both the conditions (component 821 and 822) are for the same health problem—‘Exacerbation of Asthma’. Hence, instead of displaying two separate conditions, they are grouped into one condition (component 850). From this condition, user would be able to navigate to related encounter records—in this case Encounter1 and Encounter2. Each encounter record will have the option to further navigate to related medication and observation resources. From Encounter record, users would be able to know what all medications were prescribed and what observations were made.

In the given example, the pediatric patient had been admitted to the hospital two times in ER (emergency room) due to asthma flareup. Encounter2's detail show that patient was given medication dexamethasone followed by prednisolone on next day. As per health guidelines, taking prednisolone and dexamethasone together is not advisable. Dexamethasone is a long-acting glucocorticoid and is six times more potent than prednisolone. Prednisolone is shorter acting, with a half-life. The previous encounter Encounter1 shows that medications given in a similar situation in the past were—albuterol followed by prednisolone next day. And this is consistent with treatments given to the patient in the past (other records not shown here). This indicates that medication treatment for dexamethasone followed by prednisolone is not a safe and recommended way, and the patient should have been treated with safer and tested option of albuterol followed by prednisolone.

It Is unusual for a patient to carry all the documents for follow up visit with pulmonologist plus a person unfamiliar with medical field would not understand medication names. If medication details for the given encounters were available for the pulmonologist to review during follow-up meeting, they would have flagged the Encounter2 treatment and suggested that the ER doctor should have given albuterol and prednisolone as more effective treatment always done in the past for the patient. Also, if there was a trail of all the ER visits and treatments available to the practitioner2 at the time of Encounter2, it would have hinted him/her to give safer and tested treatment and avoid risk of possible complications.

FIG. 8C shows ‘Medication View’ i.e., the way in which information can be displayed when user navigates to Medication tab in presentation layer.

Resource Linking unit lists distinct medications given to the patient. Since MedicationRequest2 827 and MedicationRequest4 828 both represent the same medication ‘prednisolone’, they are grouped into one medication record 860 while displaying on the presentation layer, on one or more computer monitors. From each medication record user can navigate to respective encounters and conditions for which the medication was given. In this case, from medication record specific to ‘prednisolone’, user can access both the hospital visits—Encounter1 823 and Encounter2 824 during which the medication was prescribed. Also, from each medication, user can see what conditions the medication was prescribed for. In this scenario, as described in FIG. 8B, even though for the display purpose, the conditions were grouped into one, original condition records are still maintained and MedicationRequest records still do refer to the exact FHIR (trademarked) condition resource to keep the data connections accurate.

FIG. 8D shows ‘Observation View’ i.e., the way in which information can be displayed when user navigates to Vital Observations tab in presentation layer such as shown on one or more computer monitors.

Unified Health Record 106 lists distinct entries of each observation of certain type. In this case, vital observation ‘SPO2’ lists two recordings—Observation1 829 and Observation2 830. From each observation resource, the user can also get details about the related encounter.

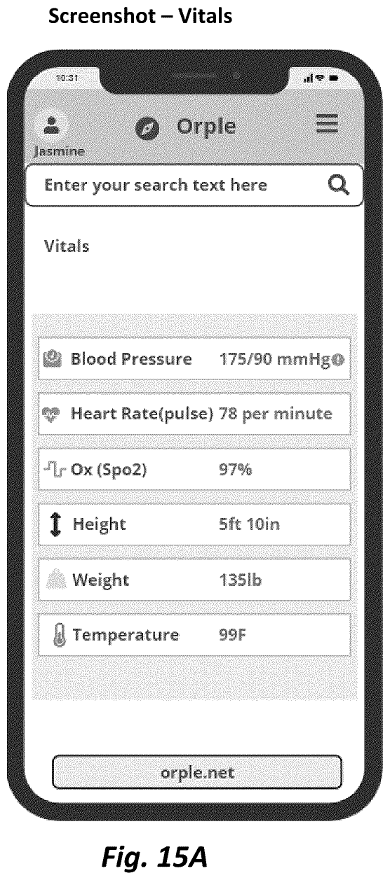

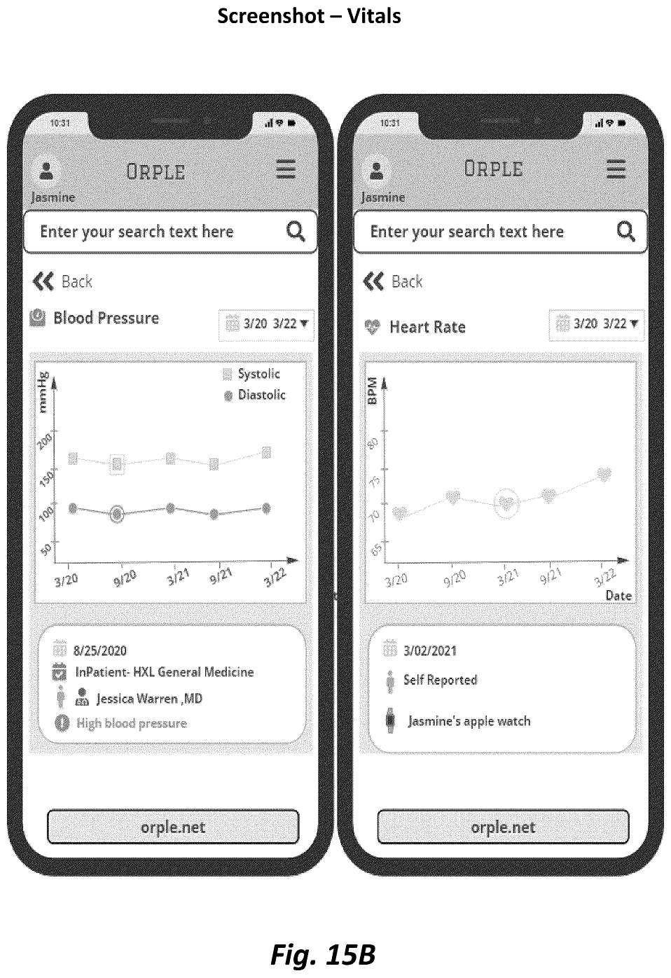

FIG. 15 shows a screenshot of possible user interface for the Vital observation type. It is described in more detail further in this disclosure.

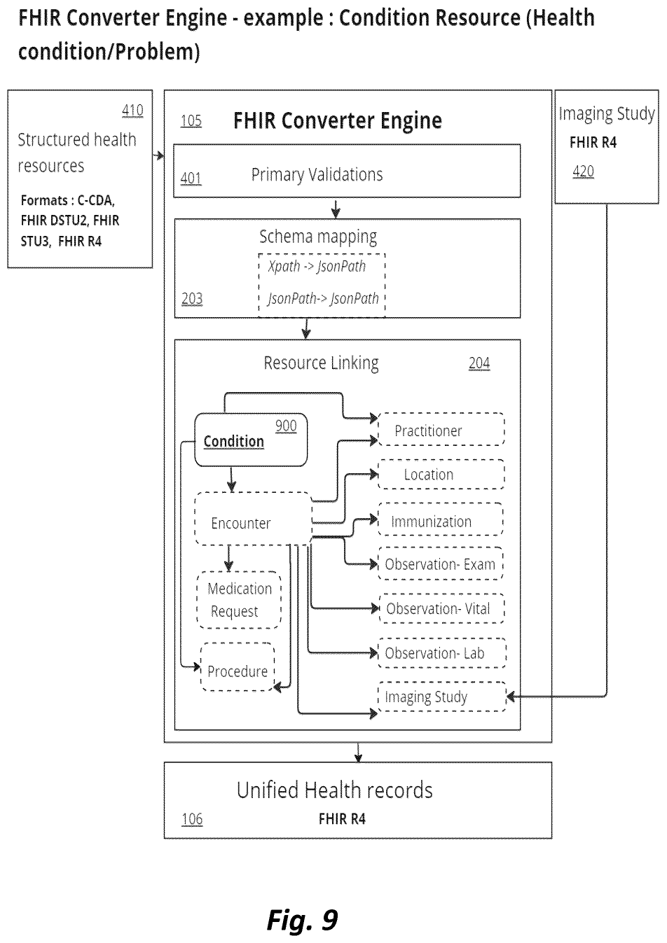

FIG. 9 shows how data is processed inside FHIR (trademarked) converter engine 105 with an example of ‘Condition’ resource. Condition or Health Condition both refer to FHIR (trademarked) resource-Condition which represents health concern or problem. In actual implementation, there could be variations in the syntax and configurations to enhance the accuracy and cover maximum data.

### Component 401 Primary Validations:

For each resource type, distinct validations are performed to ensure correct data processing. A few example validations for condition resource.

Verify if mentioned careprovider already present.

Verify if a related encounter exists.

Check if a condition resource with same name, date, patientId and Data source Type present.

### Component 203 Schema Mapping:

Attributes/field values are linked between resources of different schemas/formats based on the pre-defined schema mapping rules. A few examples:

When incoming condition data is in DSTU2 format, practitioner's name is read from JsonPath [Resource/asserter/display]. This is determined by referring to the following DSTU2 JsonPath to R4 JSON schema mapping rule:

| | | | Target | | |
|---|---|---|---|---|---|
| Resource | Field | SourceType | Type | SourcePath | TargetPath |
| Condition | PractitionerName | DSTU2 | R4 | Resource/ | Resource/ |
| | | | | asserter/display | recorder/display |

When incoming condition data is in C-CDA format, practitioner's name is read from following XPath: performer[assignedEntity/assignedPerson/name]. This is determined by referring to the following CCDA xml to R4 Json schema mapping rule:

| | | Source | Target | | |
|---|---|---|---|---|---|
| Resource | Field | Type | Type | SourcePath | TargetPath |
| Condition | Practitioner | C-CDA | R4 | assignedEntity/ | Resource/ |
| | Name | | | assignedPerson/name | recorder/display |

### Component 204 Resource Linking:

In a patient's health history certain health condition might be present for short or long period of time. Also, for each health concern/condition, a patient might have seen one or many care providers with corresponding one or more visits (encounter). As part of FHIR (trademarked) Converter engine 105, Condition resource is linked to careprovider, procedure and encounter resources. This enables users to see details of all the treatments received for the health condition including but not limited to doctor office visits, care providers involved including doctors and nurses, care locations radiologists, medications prescribed, procedures done/planned, lab results, vitals recorded, exam observations, immunizations, doctor's notes, source prescription, imaging study with DICOM (trademarked) images etc.

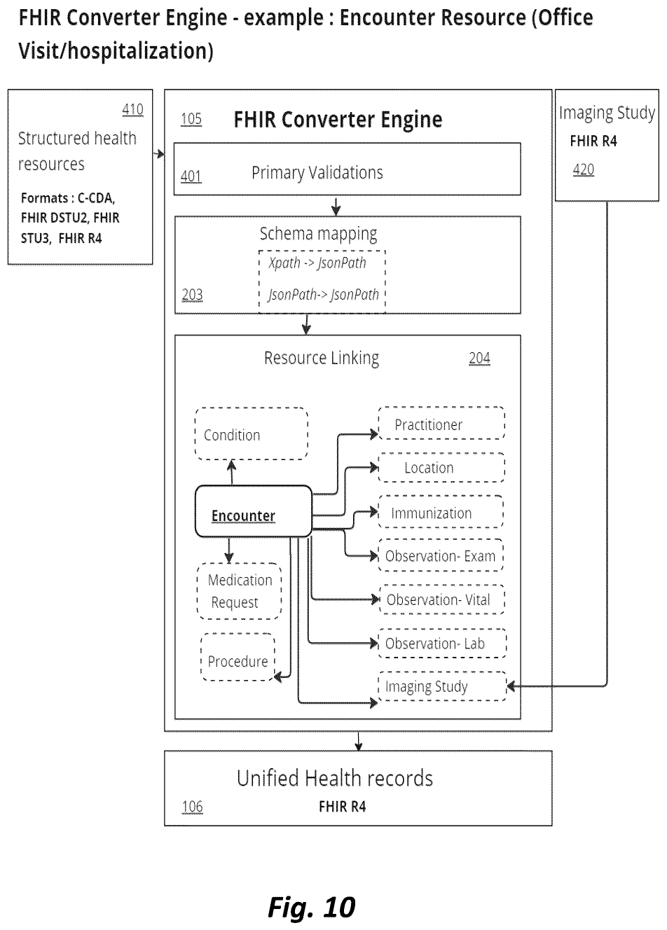

FIG. 10 shows how data is processed inside FHIR (trademarked) converter engine 105 with an example of Encounter resource. In actual implementation, there could be variations in the syntax and configurations to enhance the accuracy and cover maximum data.

### Component 401 Primary Validations:

For each resource type, distinct validations are performed to ensure correct data processing. Few example validations for Encounter resource:

Verify if encounter with same type, date, provider, and location already exists. If a match is found, link resources to the existing encounter resource. Otherwise, create new encounter resource.

Verify if encounter has already taken place in the past or planned in future. Provide appropriate indicator on the User interface.

### Component 203 Schema Mapping:

Attributes/field values are linked between resources of different schemas/formats based on the pre-defined schema mapping rules. An example:

When incoming encounter data is in C-CDA xml format, practitioner name is read from following xpath: [ClinicalDocument/componentOf/encompassingEncounter/encounterParticipant/assignedEntity/assignedPerson/name]

This is determined by referring to the following CCDA xml to R4 Json schema mapping rule:

| | | Source | Target | | |
|---|---|---|---|---|---|
| Resource | Field | Type | Type | SourcePath | TargetPath |
| Encounter | Practitioner | C-CDA | R4 | ClinicalDocument/componentOf/ | Resource/ |
| | Name | | | encompassingEncounter/ | participant/ |
| | | | | encounterParticipant/assignedEntity/ | individual/display |
| | | | | assignedPerson/name | |

### Component 204 Resource Linking:

Encounter resource is referenced by almost all the FHIR (trademarked) resources in health care management process specific to an individual. However, the current definition of encounter resource does not have a provision to specify references to all relevant resources like imaging done or medications prescribed. Resource linking unit 204 establishes links between encounter resource and all other relevant resource types. This greatly helps in navigating to desired pieces of information when accessing the encounter resource from presentation layer. Resource linking unit 204 shows that encounter resource is linked to various resources like condition, medication, practitioner, imagingStudy etc.

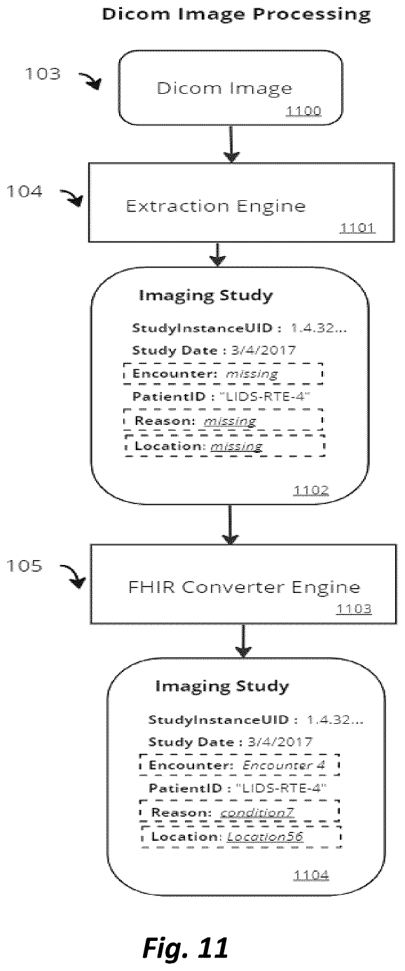

FIG. 11 describes how DICOM (trademarked) images are processed in the HealthRecord Application. A patient's health record is incomplete without radiology imaging data. Clinical records can be obtained from a variety of sources, however even today, access to imaging data is very limited. It is important that care providers can see the actual DICOM (trademarked) images along with radiology reports in addition to a patient's clinical records to provide effective, safe, accurate and evidence-based treatment. Inability to present imaging data along with clinical data creates a gap in the information which may lead to incorrect diagnosis and treatment. This may also lead to repeat scanning which puts financial stress on the patient and in some cases, exposes patient to unnecessary harmful radiations. Radiology service providers store and maintain radiology images in PACS system. The only way these images are directly accessible to care providers is through access to the PACS system. Today very few care providers have such provision available. This leaves a patient with only one option i.e., delivering the CD/DVD containing DICOM images and radiology report to the care provider's office. In addition to this there are additional challenges in this process. A care provider would be able to view DICOM (trademarked) image from the CD/DVD only if they have a computer device in the office enabled with a CD/DVD reader. Not all doctors have such computing devices available in their office. This leads to a situation where the patient must rely on radiologist's report for the diagnosis and convey the findings through the paper report given by the radiologist.

Over the period of several years a patient might have acquired a few CD/DVD disks. Carrying and maintaining these devices adds stress and extra overhead for a patient.

HealthRecord Application provides capabilities to users where they can upload imaging data in the form of radiology report and DICOM (trademarked) images 103. Reports are scanned and go through text extraction process 104. DICOM (trademarked) images are stored in long term, scalable, secure, and globally accessible storage medium, such as computer memory. These images go through DICOM processing unit described in FIG. 11.

Any DICOM image first passes through extraction engine 104 where its metadata and other key information is extracted. The information extracted includes but not limited to series details, patient details, modality, encounter, and condition details etc. Also, information related to image display like pixel spacing, image position, image orientation etc. is extracted.

All this extracted information is represented through a FHIR (trademarked) resource—Imaging Study (represented by component 1102 in FIG. 11).

In the example as shown in component 1102, the imagingStudy record may not have all the necessary references to relevant resources e.g., missing encounter link or no information about location and reason for which imaging was done.

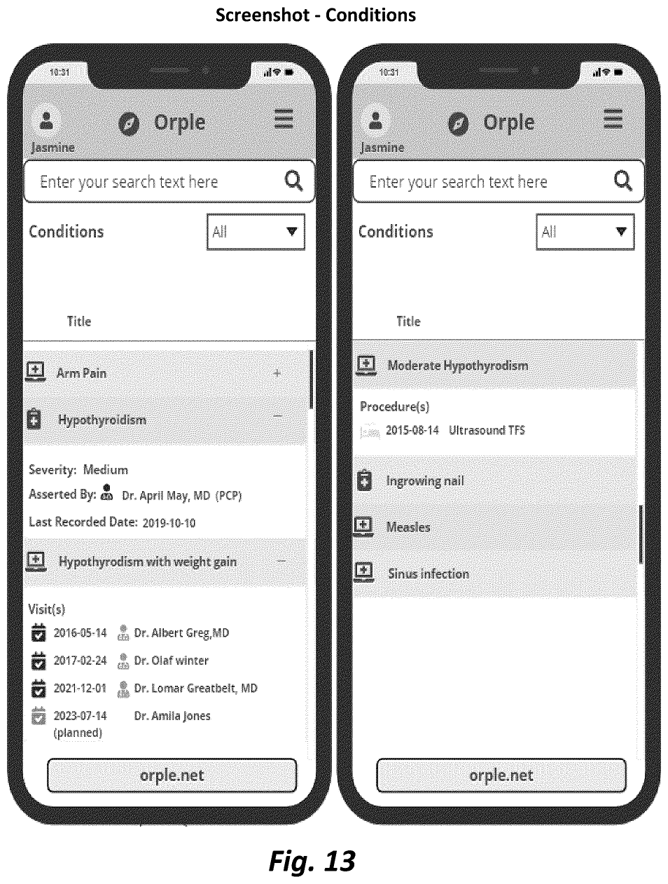

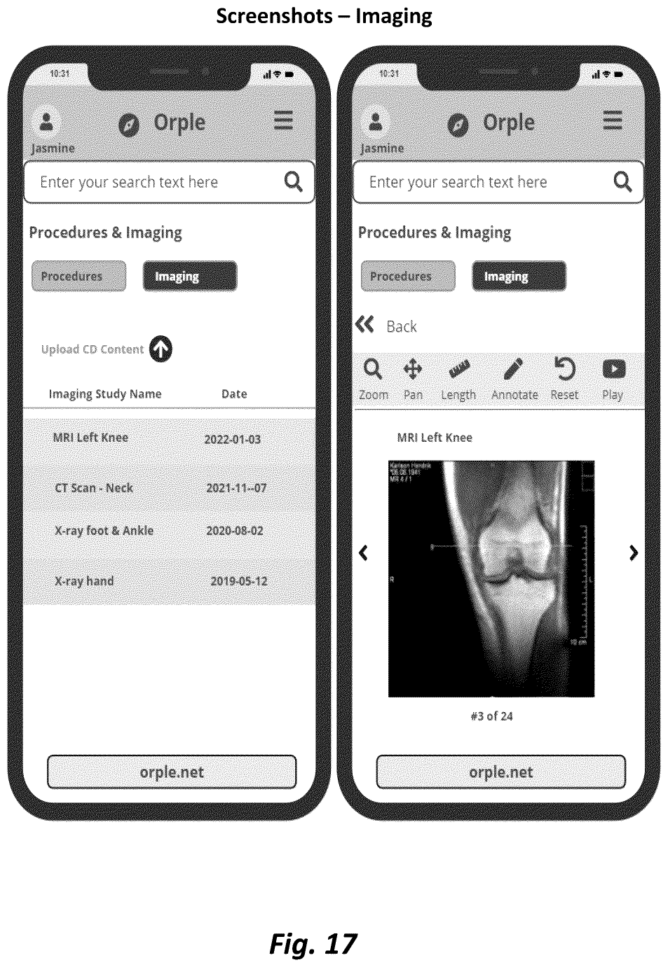

The next step—FHIR (trademarked) converter engine 105 processes the imaging study and establishes necessary data connections between imagingStudy—encounter, condition, and location. The outcome of the process is displayed in component 1104. The updated imagingStudy resource becomes part of unified health record 106, and is stored in computer memory. This record enables the imaging study to become accessible through relevant resources in User Interface 107 on a computer monitor. In this way, a user can easily access details of all the imaging work done in the past along with all the clinical data specific to a health condition from a single platform. FIG. 17 shows a possible user interface design for accessing imaging data. FIGS. 12 and 13 show how imaging data can be accessed from relevant encounter and condition records, respectively.

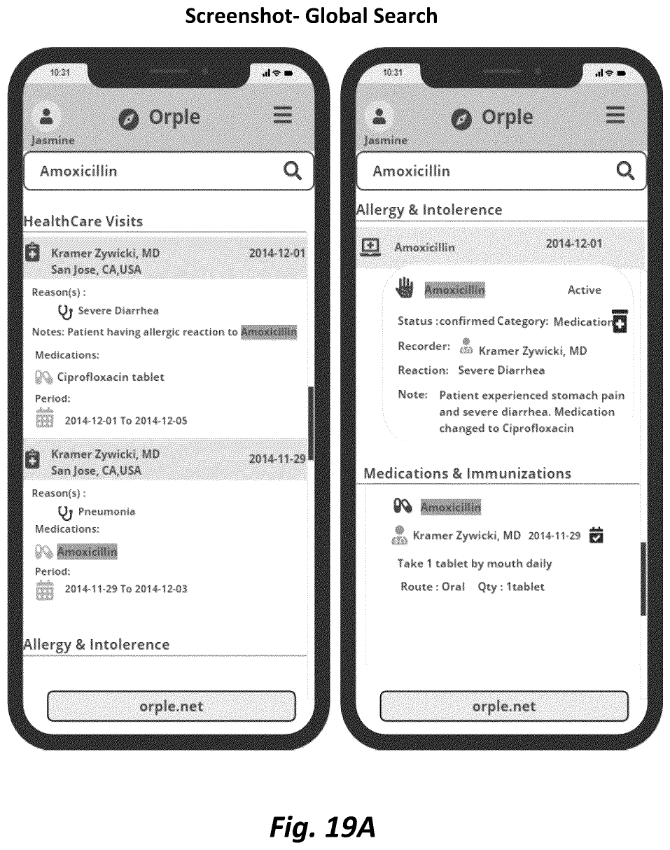

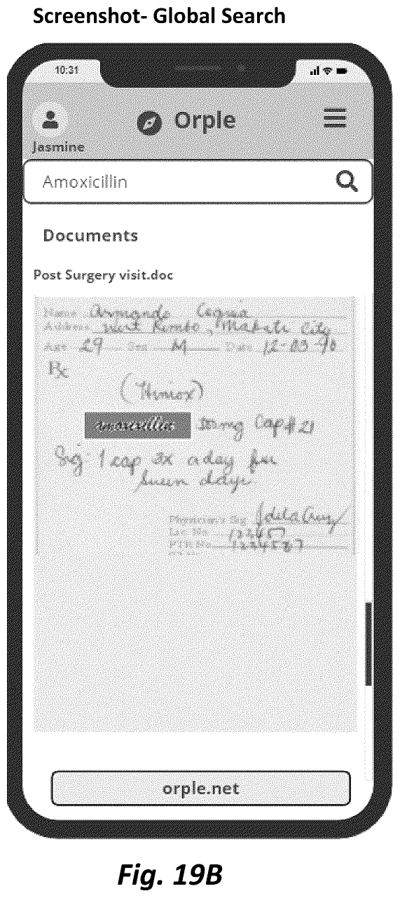

Even though health records for a particular patient are available through various resources, sometimes it becomes greatly helpful if user can enter a search text and be able to fetch all records related to the text in single click. The HealthRecord Application is configured to provide an important feature ‘Global Search’ enabling a user to access all relevant pieces of information specific to a keyword/text in a single click. A patient's data could come from a provider's hard copy files or paper document. One or more embodiment of the present invention, provides a user the ability to scan paper documents, OCR the scanned document and index its content for global search. Global search feature would perform search text lookup in the unified health records and OCR content of the uploaded documents as well. The system would highlight matching keywords in the document and other search records from unified health records while displaying results on the presentation layer. A possible user interface design of Global search feature is shown in FIGS. 19A and 19B. More details are discussed further in this disclosure.

FIG. 12 shows possible user interface design of encounter related information. In the given example, for the encounter that took place on Dec. 12, 2021, user can get the details related to condition i.e., reason for which the visit took place, diagnosis, vital observations recorded. There was a blood work prescribed during this visit, which was done later, and report was received on Jan. 2, 2022. This lab report is accessible from the encounter record. Also, through the same record user can see procedures and imaging work done. By looking at the information presented, we can derive the analysis that due to hypothyroidism, the user became overweight, had developed high cholesterol, weak muscles, and increased knee pain. This eventually led to MRI scan on Knee and eventually knee replacement surgery. We can also see what medicines were prescribed and details around that.

FIG. 13 shows a possible user interface design of the Conditions tab.

In the given example, a user has conditions related to Hypothyroidism. By looking at the information presented, we can derive the analysis that the condition went through various phases over the years. It started with moderate hypothyroidism which was in relation to an ultrasound procedure done in 2015. Eventually it led to significant weight gain leading to other health problems. Patient had been to multiple practitioners for the treatment. In the interim, the patient had managed to control weight gain for a specific period during the visit of Oct. 10, 2019. The weight gain returned, leading to serious side effects—knee pain and surgery in 2021. The user has a new planned appointment with a practitioner on Jul. 14, 2023.

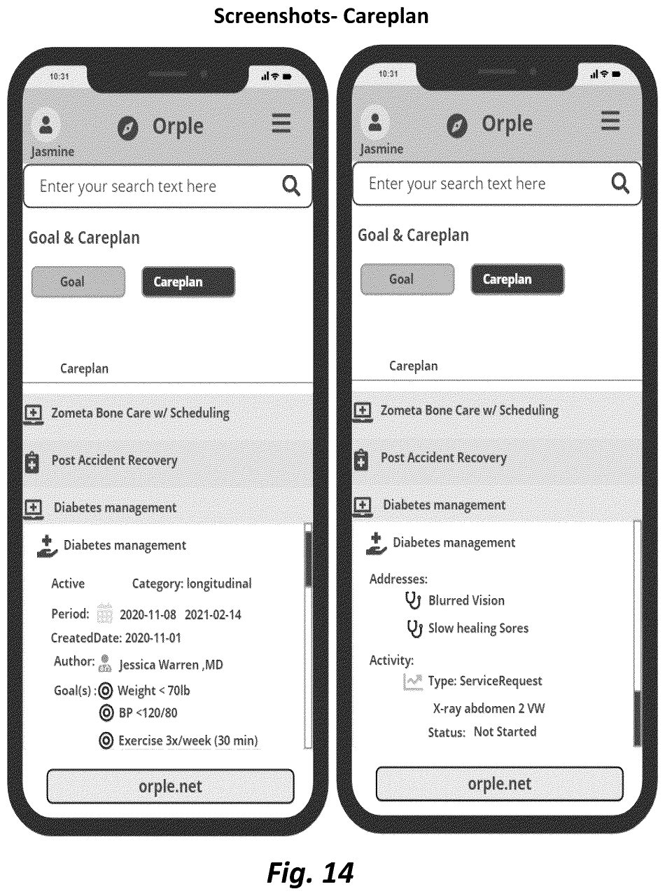

FIG. 14 shows a possible user interface design of Careplan tab. In the given example, Careplan for ‘Diabetes management’ has information about time period, category, goals, and status. It also provides reference to the practitioner record who has authored the plan. It lists goals to be achieved as part of the plan. The Careplan resource also refers to conditions it aims to address. In this case the conditions are blurred vision and slow healing sores. The Careplan also refers to imaging activities prescribed like X-ray abdomen.

FIGS. 15A and 15B show screenshots of a possible user interface for the vitals observation tab. All the screenshots and user interface designs may be tweaked to accommodate better user experience and functionality.

FIG. 15A lists all the vital types. In FIG. 15B, first screen from left shows possibly how data points can be shown for a specific vital—blood pressure. By selecting a particular data point, a user can see more details like the source of the data, date when data was collected, encounter if any specific to it, practitioner involved and warning flag if any. Similarly, second screen on the right side shows sample design for heartrate data collected.

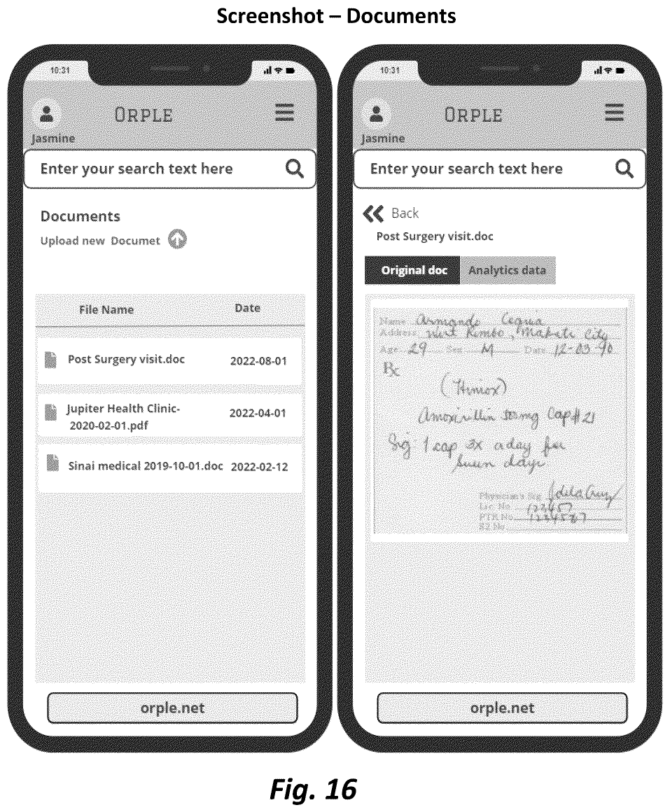

A user can upload health record related documents in scanned format supporting various file types including but not limited to JPEG/JPG, PNG, BMP, TIFF, PDF, Word (DOCX), Excel (XLS), PowerPoint (PPT), and HTML etc. These documents can be printed records obtained from healthcare organizations, laboratory reports or even handwritten prescriptions. After upload, they are further processed through extraction engine and FHIR (trademarked) converter engine 105. A user can access original copies of the documents through Documents section on the user interface. FIG. 16 shows an example of possible user Interface of documents section. As seen in FIG. 16, an original copy of the prescription is displayed to the user.

FIG. 17 shows a possible user interface for display on a computer monitor for the Imaging section. The system designed as a part of one or more embodiments of the present invention gives a user the ability to upload a radiology imaging related report and DICOM (trademarked) images from the user interface. After passing through the extraction engine 104 and FHIR (trademarked) converter engine 105, these records result into imaging study FHIR (trademarked) resources. Information related to respective encounter, condition, location, practitioner etc. can be viewed through the user interface for the selected Imaging study. Also, for each imaging study, a set of related high quality DICOM (trademarked) images are available for viewing and annotating. These images are displayed through a customized DICOM (trademarked) viewer.

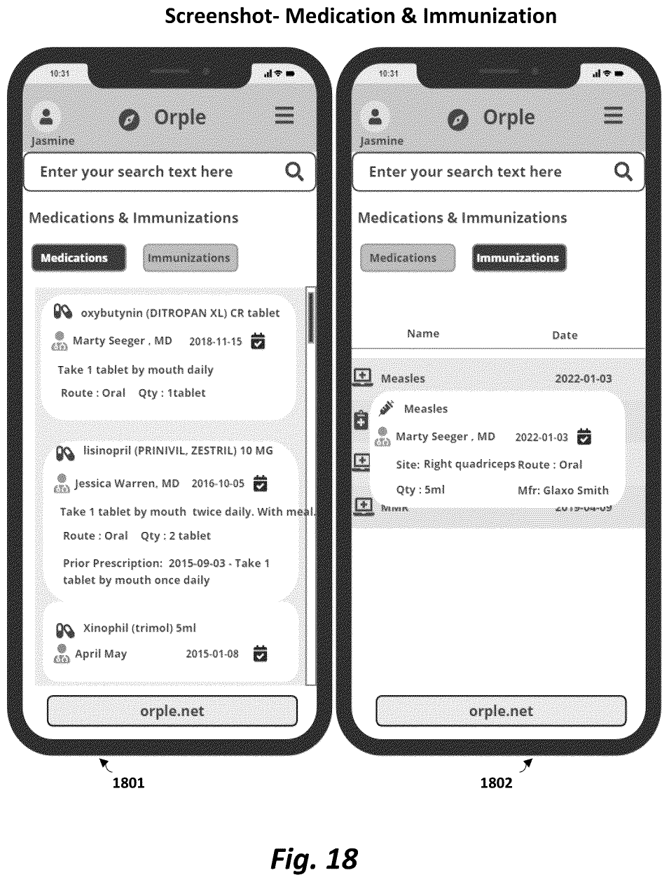

FIG. 18 shows possible user interface design for medication & immunization. As shown in component 1801, medication records can show references to relevant resources like encounter, practitioner, condition etc. They can also display additional information regarding the prescription including dosage instructions, route, quantity, prior prescription etc. component 1802 shows possible user interface design for immunization records. Immunization record may provide reference to practitioner, site, encounter, quantity, manufacturer etc.

FIGS. 19A and 19B describe a global search feature. In the example scenario, a patient has been to doctor's office for some health concern. A doctor thinks of prescribing medicine: ‘Amoxicillin’. However, the doctor also wants to know all the past events when a patient had taken the specific medication. Using the feature global search, a user can enter key text ‘Amoxicillin’ in the search text box. The global search feature will scan through all the clinical records and document copies uploaded into computer memory. Results are presented to a user where five different areas were found to have the reference to the search text ‘Amoxicillin’. First, on Nov. 11, 2014 during an encounter with Dr. Kramer Zywiki, the patient was prescribed medicine—Amoxicillin to treat pneumonia. This is indicated through the encounter and medication resources. However, in two days, the patient developed an allergic reaction to the medication and had a follow up with the doctor on Dec. 1, 2014. Encounter for the follow-up visit mentions allergic reaction to the medication in the notes section. Also, the allergy and intolerance section show the allergy record for amoxicillin. The document section on the user Interface shows a scanned copy of a prescription prescribed more than twenty years ago when the patient was given the same medication. All these search results highlight the search key text so that a user can clearly locate the record.

Today, we are becoming global citizens. People move across places. Even across countries. Digital and paper records generated in one place may not be available when needed at a certain place. It is important to have a platform giving a user the ability to see health information data originating from various sources through a single solution. A system, apparatus and method configured through one or more embodiments of the current invention provides such a platform.

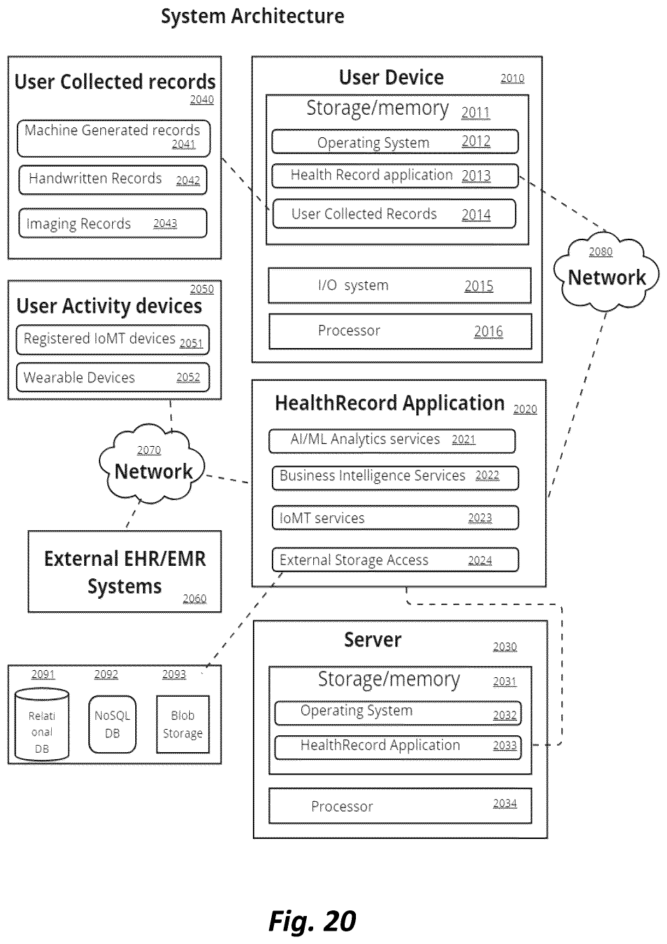

FIG. 20 shows high level design of system architecture for the system described through one or more embodiments of the present invention which can be referred to as HealthRecord Application. User device 2010 is a computing device from which a user can access the HealthRecord Application 2020. A user can be any individual having health records unified through the HealthRecord application. He/she can be an actual patient or legal guardian of the patient (in case of minor). A user device can be of any type but not limited to a Mobile device, laptop, Desktop, PDA etc. It includes storage/memory 2011 which can be persistent or non-volatile memory to store an in-memory data or independent storage device such as hard disk drive or solid-state drive. It may also contain operating system 2012. A user device is configured to access health record application 2013 from the operating system 2012. The HealthRecord application is hosted externally on devices/server 2030 accessible over network. A user device also includes processor 2016, I/O system 2015 such as but not limited to a computer mouse, computer keyboard, speakers, display screen, pen etc.

HealthRecord application 2020 can be accessed from the User Device 2010 as software as a service (SaaS) application over network 2080. In some examples, network 2080 may be any one of different types of networks such as the Internet—cellular network, cable network, Wi-Fi network. User Device 2010 also may contain user collected records 2014.

User Collected record 2014 may be sourced from various ways including—machine generated records 2041. Machine generated records 2041 can be digital copies of health records or scanned copies of the previously printed documents. Another type of user generated records are handwritten records 2042 such as but not limited to prescription or doctor's notes. User Collected records could also contain radiology images such as DICOM (trademarked) images 2043. All these records 2040 can be submitted to HealthRecord application 2020 from User Device 2010 via network 2080.

HealthRecord Application 2020 can be hosted on one or many external server(s) 2030. HealthRecord application may receive data from various external sources. It can receive data from EHR i.e., Electronic Health Record System or EMR i.e., Electronic Medical record System 2060 via network 2070 through API—Application Programming Interface. HealthRecord Application 2020 may also receive data from external devices including user wearable devices capturing health data such as but not limited to smart watch, pedometer etc. HealthRecord Application 2020 can also receive data from registered IoMT—Internet of Medical Things devices 2051, specially designed to capture health telemetry from a patient. These user activity devices 2050 can communicate with HealthRecord application via network 2070. In some examples, network 2070 may be any one of different types of networks such as the Internet—cellular network, cable network, Wi-Fi network etc.

HealthRecord application 2020 contains various services responsible for providing different types of functionalities such as but not limited to AI/ML Analytics service 2021, Business Intelligence Services 2022, IoMT services 2023 etc. HealthRecord application 2020 can save data to external storage mediums such as relational Database 2091 which could be SQL server or to NOSQL database 2092 which could be Cosmos DB. It can also store scanned images, digital copies of records, large files such as DICOM (trademarked) images to external long term secure storage such as Blob storage 2093.

HealthRecord Application can be hosted on remotely accessible computing devices called server 2030. Server 2030 will host the HealthRecord application 2020. Server's hardware would include processor 2034, storage/memory 2031 which can be persistent or non-volatile memory to store an in-memory data or independent storage device such as hard disk drive or solid-state drive and operating system 2032.

### Use Cases

This section describes some possible scenarios where system designed through the invention can be useful.
(1) In an example scenario a patient has pain in the knee for couple of months. The patient visits Primary care physician (PCP). PCP prescribes X-ray. Meanwhile the pain becomes worse. Due to unavailability of the PCP (long wait-time for next appointment due to shortage of doctors in their group), patient decides to see specialist orthopedic giving quick appointment. An orthopedic doctor without thorough examination predicts fracture and sends for X-ray. X-ray exam has no findings. Patient goes back to the Orthopedic doctor. Orthopedic doctor predicts possibility of infection and gives antibiotics/predicts strain and gives strong pain killers for a week. After the medication dose is finished, there is no improvement in the patient's condition. Orthopedic predicts tumor and asks for MRI. Meanwhile Patient sees another orthopedic doctor for second opinion. Second orthopedic doctor rules out tumor, predicts fracture/major ligament tear and prescribes CT and sends to surgeon for surgery (without any imaging evidence). Meanwhile, first orthopedic doctor's team has obtained insurance authorization for MRI to diagnose possible tumor. When second orthopedic doctor's office reached out to insurance for CT scan authorization, insurance denies the CT authorization. Third party company (delegated to for pre-auth work by insurance) has a doctor who does not have access to the patient's entire health history and detailed reasons, recommends that MRI is needed first as per predefined guidelines. Now a patient cannot proceed with second orthopedic doctor's treatment. Patient decides to start afresh and get referral for a good specialist from PCP. Again, due to unavailability of original PCP's in-person appointment dates in near future, patient decides to go to new PCP. This time the new PCP says he/she cannot proceed without seeing all the records from all the doctors. Patient provides previous blood work thru laboratory website, Xray from radiology portal. But does not have a way to show all the notes and radiology images from PCP, Orthopedic doctors in one place and convey their justifications. Patient should not have to wait for the care provider's EHR to be able to connect to all other care provider's EHRs and fetch data, waste multiple appointments in getting appropriate data fetched, integrated, and interpreted. A system, apparatus and method configured through one or more embodiments of the current invention can be very helpful in addressing patient's data needs in this case.
(2) Data collected by one or more embodiments of the present invention, is configured to result in a population health data pool which can be useful for researchers or health professionals. An example scenario could be where a research team/doctor's team needs to get data satisfying following condition: Get us details of all medications, procedures, lab reports done for patients along with their CT scan and MRI scan reports from last five years who are diagnosed with Leukemia in last two years and who are between the age of thirty to forty years old. Such queries normally would require long wait time and interaction with multiple systems. By the time results arrive, patient's health may have deteriorated. A system configured through one or more embodiments of the present invention (HealthRecord Application) allows sharing of health data with a user's permission. This data can be anonymized based on user preferences. Using data collected by HealthRecord Application, such information can be obtained quickly within few seconds. Possible logic could be—get the list of all patients having age between thirty to forty years, with a health condition ‘Leukemia’ with confirmation date within last two years. Get encounters related to these condition records. From each encounter, get details of medication prescribed, changes in medications, doctor's notes, observations made, vitals monitored, laboratory reports, procedures done, radiology imaging tests done. This will allow access of data not only from EHR/EMR systems received in digital format but also from paper documents and radiology images.

This can also open an opportunity to access data from a global health population. Helping physicians get insight into treatments done in different countries and researchers could evaluate patterns in health condition progression in people from dispersed geographical locations.
(3) A thirty-year-old female suffering from repeat hospitalization due to respiratory illness. Possibility of viral and bacterial infections ruled out. Doctor's need to come up with accurate diagnosis and determine effective Careplan to avoid hospital re-admission. The patient could use HealthRecord application to collect data from various sources. Latest health data may be collected from EHR/EMR systems and IoMT device as digital records. The patient may have paper copies of the prescriptions obtained during doctor's visit over the period of last several years. Patient can also ask parents or guardians to provide medical records if any from the years when patient was a minor. Most of these records would be handwritten notes, prescriptions. A parent/guardian can share photos of these records via mobile device and patient can upload those into the system. Also, a patient can request hospitals and doctor's office from where treatments were received over past several years to provide paper copies of the records and upload images of the documents into the HealthRecord Application system. This will help a patient create repository of all records in one place. A doctor at the latest hospital ER visit can review all the data and see that patient had suffered from acute respiratory infections during childhood. Also, a patient had been taking steroids for several years from the age of six to eleven to control lung inflammation. This helps the doctor confirm the diagnosis as Asthma and decide correct medications to avoid flareups in future.

In case a user wishes to exit the system and no longer use it, they would have an ability to export all the unified medical records and the analytics. So that it will be useful for their future use and parents can pass on to future generation their health history in hard copy/paper format.
(4) One or more embodiments of the current invention can also be used to generate unified health records for veterinary services.
(5) Today, several health conditions still do not have effective treatment or a permanent cure. One of the reasons is a lack of longitudinal population health data. One such case is Asthma where millions of patients suffering must rely on daily medications which contribute to significant side effects over long-term use. If unified health records for asthmatic patients covering various life stages such as infants, children and adults are available along with spirometry test reports, lung function test report, medication history, doctors visit, X-ray images, this can help researchers get valuable insights and progress the research.
(6) An international tourist enjoying a vacation on a beach in a foreign country suddenly develops extreme knee pain and is unable to perform routine activities, and as a result visits a hospital Emergency room. The patient mentions to the care provider at the hospital about a recent fall in their home country two months ago for which a CT scan was performed. The patient can access the CT scan report and show the DICOM (trademarked) images to the doctor in hospital where a doctor identifies a ligament tear which was overlooked by the radiologist at the time of an exam in the home country. The patient had been asymptomatic after taking pain medications for a few weeks after the fall two months ago and suddenly developed pain while on vacation due to increased activity. All this diagnosis can be deduced by reviewing a unified health record of the patient through a system created by one or more embodiments of the current invention. Plus, it will avoid unnecessary exposure to radiations by eliminating the need of another CT-scan.
(7) An eighty-year-old man suffering from kidney disease has moved back to his home country after spending a significant portion of his life in other countries. The patient is waiting outside a doctor's office with all the notes, prescriptions, CD/DVD disks in two huge binders. Also, the patient has noted down references to several EHR/EMR systems having a portion of his health record to show to the doctor. There is a long line of patients outside the doctor's office and the doctor may have maximum twenty minutes to give to the patient. It would be unrealistic to expect the doctor to be able to derive meaningful information from the records in the binders, several EHR/EMR systems and radiology CD/DVD/disks. Also, it adds tremendous stress to the patient in carrying all of this information for every doctor visit. Instead, the patient could access and share all the information through unified health records by using a system of one or more embodiments of the present invention.

A system created through one or more embodiments of the present invention provides a user with all the wellness information since the beginning of life. If available, it can also include details related to fetal stage (prenatal period) obtained from an Ob-GYN. This will give a wholistic view of the user's health history, show patterns, projections and contribute to research by providing health records across various stages of life.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. A program executable by at least one processor of a computer device, the program comprising sets of instructions for:
receiving, at the computer device, a first plurality of personal health records for a specific individual from a corresponding first plurality of sources via a network, wherein each of the first plurality of personal health records includes a first plurality of fast healthcare interoperability resources (FHIR) and a first plurality of Consolidated Clinical Document Architecture (C-CDA) resources, wherein the first plurality of FHIR resources are in a corresponding plurality of FHIR format versions;
receiving, at a client device, a second plurality of personal health records for the specific individual from a corresponding second plurality of sources in a plurality of formats other than FHIR format;
processing one or more of the first plurality of FHIR resources and the first plurality of C-CDA resources to form a processed second plurality of FHIR resources, so that all of the processed second plurality of FHIR resources are in a target version of FHIR format, wherein the processed second plurality of FHIR resources are a first set of data;
extracting a third plurality of FHIR resources in the target version of FHIR format from the second plurality of personal health records, where the third plurality of FHIR resources are a second set of data;
establishing references in computer memory between one or more FHIR resources of the first set of data and one or more FHIR resources of the second set of data; and
changing one or more values, in one or more data fields of one or more FHIR resources of the first set of data and one or more FHIR resources of the second set of data;
wherein the steps of establishing references and changing one or more values, result in a fourth plurality of FHIR Resources that are in the target version of FHIR format;

2. The program of claim 1 further comprises sets of instructions for:
generating, at the client device, a user interface;
displaying the user interface on a computer monitor, wherein the user interface includes a plurality of user controls, and each of the plurality of user controls includes a plurality of data fields, wherein each of the plurality of user controls relates to a specific FHIR type;
on the user interface, enabling selection of a first user control of the plurality of user controls;
on the user interface, enabling selection of a first data field of the plurality of data fields of the first user control; and
responding to the selection of the first data field of the first user control by displaying a set of information on the computer monitor, wherein the set of information includes a plurality of data elements of a corresponding plurality of FHIR types related to the specific individual.

3. The program of claim 1 wherein
the said receiving of the second plurality of personal health records and extracting the third plurality of FHIR resources in the target version of FHIR format, further comprises sets of instructions for:
receiving at the client device, a plurality of Digital Imaging and Communications in Medicine (DICOM) images related to the specific individual;
extracting image data in a plurality of key value pairs from each of the plurality of DICOM images;
generating an imaging study FHIR resource in the target version of FHIR format for the image data; and
displaying the plurality of DICOM images through an image viewer in the said user interface.

4. The program of claim 1, further comprising sets of instructions for:
enabling input of a search text in a user control, on the said user interface;
searching for the search text in the fourth plurality of FHIR resources related to the specific individual, wherein the step of searching results in a fifth plurality of FHIR resources, and wherein the fifth plurality of FHIR resources are a search result;
displaying the search result on the user interface, in a plurality of user controls, wherein
each of the plurality of user controls relates to a specific FHIR type; and
highlighting the search text in the displayed search result on the user interface.

5. The program of claim 1, further comprising sets of instructions for:
generating an Application Programming Interface (API);
wherein the API enables exporting of a plurality of personal health records from the fourth plurality of FHIR resources in a desired version of FHIR format;
and wherein the API enables a consumer to access aggregated and updated FHIR resources generated from originally disconnected and incomplete personal health records.

6. The program of claim 1, further comprising sets of instructions for:
displaying in a chart user control, on the said user interface, a plurality of values for a Vital Observation FHIR resource of a specific type, related to the specific individual;
enabling in the chart user control, selection of each of the plurality of values for the Vital Observation FHIR resource of the specific type; and
wherein selection of a value of the plurality of values for the Vital Observation FHIR resource of the specific type, results in display of information in a user control on the said user interface, from one or more related FHIR resources for the specific individual.

7. The program of claim 1, further comprising sets of instructions for:
displaying in a chart user control, on the said user interface, a plurality of values for a Lab Observation FHIR resource of a specific type, related to the specific individual;
enabling in the chart user control, selection of each of the plurality of values for the Lab Observation FHIR resource of the specific type; and
wherein selection of a value of the plurality of values for the Lab Observation FHIR resource of the specific type, results in display of information in a user control on the said user interface, from one or more related FHIR resources for the specific individual.

8. The program of claim 1, wherein
the first plurality of sources includes at least one of a group consisting of: an electronic Health Record System (EHR), a wearable device, and an IoMT device.

9. The program of claim 1, wherein
the second plurality of sources includes at least one of a group consisting of:
a paper copy from doctor's office,
a printed paper received from healthcare organization,
a handwritten script,
a laboratory report,
an imaging report, and
a Dicom image.

10. The program of claim 1 wherein
each of the first plurality of personal health records is a computer readable file selected from the group consisting of an XML file and a JSON file.

11. The said program of claim 1 wherein
each of the second plurality of personal health records is a computer readable file selected from a group consisting of a JPEG/JPG file, a Dicom file, a PNG file, a BMP file, a TIFF file, a PDF file, a Word (DOCX) file, an Excel (XLS) file, a PowerPoint (PPT) file, and an HTML file.
